(19) United States
(12) Patent Application Publication
(10) Pub. No.: US 2004/0121405 A1
(43) Pub. Date: Jun. 24, 2004

(54) Selective covalent-binding compounds having therapeutic diagnostic and analytical applications

(76) Inventor: Bernard S. Green, Yavne (IL)

Correspondence Address: Anthony Castorina, G E Ehrlich, Suite 207, 2001 Jefferson Davis Highway, Arlington, VA, 22202, US

(21) Appl. No.: 10/474,042

(22) PCT Filed: Apr. 16, 2002
(86) PCT No.: PCT/IL02/00307

(51) Int. Cl. G01N 33/53; C07K 16/46; C08B 37/00

(52) U.S. Cl. 435/7.1; 530/387.1; 530/409; 536/53

(57) ABSTRACT

Novel compounds are provided having enhanced affinity for a desired, preselected, target substance (a small molecule; a macromolecule such as a protein, a carbohydrate, a nucleic acid, a cell, a viral particle, etc.) by modification with chemical groups that allow these substances to form strong bonds, such as irreversible covalent bonds, with the desired target substance. These qualities of tight, specific binding are reminiscent of antibody-like affinity; hence the new substances are termed COBALT, an acronym for Covalent-Binding Antibody-Like Trap. The present invention includes a process wherein a target species is chosen and then, by synthetic chemical procedures and modifications, novel substances (COBALTs) are obtained that exhibit selective and covalent binding to the preselected target species. The applications of the COBALTs include diagnostic, analytical, therapeutic and industrial applications.

### FIELD OF THE INVENTION

The present invention pertains to compounds, herein designated COBALTs, or Covalent-Binding Antibody-Like Trapping or -Trap, characterized by specific binding to a target with antibody or antibody-like affinity for a desired, preselected, target substance (a small molecule; a macromolecule such as a protein, a carbohydrate, a nucleic acid, etc.; a cell; a viral particle; etc.) and which contain chemical groups that allow these COBALTs to form strong, specific bonds, such as irreversible covalent bonds, with the target substance for which the COBALT was specifically designed.

### BACKGROUND OF THE INVENTION

The superior selectivity of antibodies, especially monoclonal antibodies, has led to their widespread use in many biomedical and other applications such as immuno-assay diagnostics and therapeutic drugs. However, antibodies have a number of drawbacks compared to synthetic compounds including: the difficulty of optimization or modification via chemical modification; the necessity of their being maintained at low temperatures; their short shelf life (subject to thermal and microbial degradation); bio-contamination; high production costs. In addition, since antibodies are generally produced using the immune system, the genetic limitations of the animals used may restrict the binding site variability. This last restriction may be overcome by using phage-display and other genetic engineered methods for antibody production but here, too, it has not always been possible to raise effective antibodies for every desired target material. While some antibodies have very high affinity constants others do not have the degree of binding needed for a particular, desired application. As a result of these and other considerations, significant efforts have been devoted to develop antibody mimics. However, the antibody mimics produced thus far have had limited practical application. In particular, these antibody mimics suffer from one or more of the following: lack of general applicability; insufficient selectivity; low affinity constants. Thus, there is a clear need for improved antibody mimics and this invention addresses those needs by providing antibody mimics, here termed COBALTs, as described above, which have broad application to biomedical and other areas.

Biological recognition, i.e., the specific, attractive interactions between the myriad substances of nature which involve selective interactions and which are essential for life, is universally based on reversible, non-covalent interactions. Even the “essentially irreversible”, extraordinarily tight binding interaction between biotin and avidin, Ka˜10¹⁵, involves no covalent bonds. However, for many diagnostic, therapeutic and other applications, advantages would accrue to synthetic substances which exhibit antibody-like specific recognition for a given biological substance but which would bind covalently, i.e., essentially irreversibly under normal circumstances, to that selected substance.

The background art does not teach or suggest a general approach to enable the design of covalently bound ‘traps’, ‘tags’ or ‘labels’ for particular biological substances, (beyond the restricted, narrowly defined enzyme inhibitors, which form covalent bonds within active sites, or activated ligands which form covalent bonds within antibody or receptor binding sites).

### SUMMARY OF THE INVENTION

The present invention overcomes the deficiencies of the background art by providing a variety of substances which have an affinity for a desired, preselected, target substance (a small molecule; a macromolecule such as a protein, a carbohydrate, a nucleic acid, etc.; a cell; a viral particle; etc.) and which contain chemical groups that allow these substances to form strong bonds, such as irreversible covalent bonds, with the desired target substance. These substances are termed COBALTs, or Covalent-Binding Antibody-Like Trapping or -Trap, as they are characterized by tight, specific binding to a target, with antibody or antibody-like affinity. Since these substances according to the present invention preferably feature a mechanism in which a covalent bond forms specifically between the desired target and the COBALT substance, the letter “T” of COBALT may also stand for “tagging” or “tag”, as the covalent bond may cause binding between the desired target and the COBALT substance to be irreversible.

The present invention optionally and more preferably includes a method wherein a target species (as above) is chosen and then, by synthetic chemical procedures and modifications, novel substances (COBALTs) are obtained or selected from combinatorial libraries that exhibit selective and covalent binding to the preselected target species. The COBALT substances themselves may optionally be categorized according to different types of structures, including but not limited to, molecularly imprinted polymers, cyclodextrins, triazines and peptides, which may be selected from these combinatorial libraries of chemically reactive substances that can covalently react with a target substance. A molecularly imprinted polymer (MIP), as discussed in greater detail below, is typically synthesized in the presence of the target molecule, and hence is designed to bind specifically to that target molecule. The peptide derivative may include at least one of cyclic, linear and modified peptides and derivatives thereof.

The COBALT substances of the present invention are preferably designed to be highly specific, and to bind to the target substance with a high degree of specificity, regardless of the category or type of COBALT substance which is used. The uses of the COBALTs include diagnostic, analytical, therapeutic and industrial applications.

Without wishing to be limited to a single hypothesis, the binding mechanism between the COBALT and the target substance may occur as follows. An initial non-covalent complex may be pictured as forming rapidly between the COBALT and the target; other substances, different from the target substance, may be expected to either not form such complexes or alternatively to form complexes with a much shorter lifetime (in other words, their stability will be much lower) than that of the COBALT-target substance complex. The orientation of the two components, the COBALT and the target substance, in the complex is such that a chemical reaction can rapidly ensue to produce a new substance having a strong, covalent bond (or bonds) between the two initial components. Complexes formed by the COBALT and substances other than the target substance, even if they exhibit some stability, may either not have the orientation or lifetime required for covalent bond formation or the reaction rate would be expected to be far less than that between the COBALT and the desired target substance. Use of the term complex, does not rule out the possibility of more than one complex between the COBALT and the desired target substance. Indeed, for some of the applications described herein, there may be many different complexes formed, but the overall result is that the COBALTs are expected to display a preferred affinity for the desired target substance, relative to other materials, and to preferentially react chemically with the desired target substance.

Examples of situations where the chemically reactive, COBALT, approach may have important therapeutic and other applications include increased binding of a target molecule compared to conventional, noncovalently binding agents. The irreversible chemical reaction can eventually tag or trap selectively essentially all targets that initially bind, whereas with conventional, noncovalent binding, once the equilibrium constant is reached, no additional target molecules may be trapped. Illustrative potential applications include assay detection at low concentrations of the target analyte or the more effective therapeutic action of a COBALT allowing lower dosages of more effective drugs.

According to the present invention, there is provided a compound for specifically binding a molecular structure, comprising a selective and chemically reactive compound with an enhanced apparent affinity constant. Preferably, the compound is an antibody mimic. Optionally and more preferably, the compound is a molecularly imprinted polymer (MIP), the MIP being modified through chemical activation in order to react covalently with the molecular structure.

Alternatively, the target substance is a chemically reactive substance and the COBALT is designed or selected so as to selectively react with the target. An important implementation of the present invention involves MIPs designed to react covalently with reactive organophosphate toxins (OP-agents) such as DFP, soman, sarin, VX, etc.

According to one implementation of the present invention, this COBALT, which is preferably an MIP, is chemically modified by including an isocyanate, or isothiocyanate, functional group. Alternatively and preferably, the MIP is chemically modified by including an isocyanate, or isothiocyanate functional group and the molecular structure is a steroid. More preferably, the steroid is cholesterol or a bile acid. Also alternatively and preferably, the MIP contains a nucleophile such as at least one of an oxime, a hydroxylamine, a hydrazine, a phenol and a 2-iodosobenzoic acid, and so forth, for specific and tight binding to organophosphates. Also alternatively and preferably, the MIP contains two or more boronic acids or two or more aldehyde functions for specific and tight binding to carbohydrates.

Other optional functional groups which the COBALT may contain, preferably as part of the MIP implementation, include but are not limited to chloromethylphenyl and 2,5-diketo-N-phenyltriazoline or any other triazine related functional group; functional group includes at least one of an alpha-halomethyl ether, wherein a halogen moiety may be fluoro, chloro, bromo or iodo; a beta-haloethyl ether, wherein a halogen moiety may be chloro, bromo or iodo; and a halomethylaryl, wherein a halogen moiety may be fluoro, chloro, bromo, or iodo; or carboxylic acid chloride or an activated carboxylic acid (e.g., 4-nitrophenyl ester, N-hydroxysuccinimide ester, pentafluorophenyl ester, etc.). This list of functional groups is not intended to be inclusive but illustrates the scope of appropriate functional groups.

According to another embodiment of the present invention, the COBALT compound is a cyclodextrin derivative which has been chemically modified to react covalently with the target molecular structure. According to one implementation of the present invention, this COBALT, which is preferably a cyclodextrin, and more preferably is an alpha-, beta-, or gamma-cyclodextrin, includes at least one or more amino groups (replacing one or more of the hydroxyl groups). More preferably, one or more of the hydroxyl and amino groups are linked directly to arylcarboxylic acid groups, arylmethylcarboxylic acid (or other arylalkylcarboxylic acid) groups via amide or ester bonds. Also more preferably, one or more of the hydroxyl and amino groups are linked directly to aryl or arylmethyl groups, where aryl refers to, but is not limited to, phenyl and substituted phenyl, pyridyl and substituted pyridyl, naphthyl and substituted naphthyl groups.

According to yet another embodiment of the present invention, the COBALT compound is a triazine derivative being chemically modified to react covalently with the target molecular structure. According to one implementation of the present invention, this COBALT, is preferably a triazine, and more preferably is a derivative of 2,4,6-trichloro-1,3,5-triazine (cyanuric chloride) wherein one or more of the chloro groups are replaced by alcohols, phenols or preferably by amine-containing derivatives, as described, for example, by R-X Li, V. Dowd, D. J. Stewart, S. J. Burton and C. R. Lowe (1998) Nature Biotechnology 16, 190-195, and references therein.

According to still another embodiment of the present invention, the COBALT compound is a peptide or derivative thereof being chemically modified to react covalently with the target molecular structure. Preferably, the peptide derivative is at least one of cyclic, linear and modified peptides and derivatives thereof.

According to yet another embodiment of the present invention, the COBALT compound is an antibody or antibody fragment or derivative thereof, being chemically modified to react covalently with the target molecular structure.

Preferably, the chemical modification to the cyclodextrin, triazine, peptide or antibody includes introduction of one or more isothiocyanate groups. Other optional chemical modifications to the cyclodextrin, triazine, peptide or antibody include but are not limited to chloromethylphenyl and 2,5-diketo-N-phenyltriazoline or any other triazine related functional group; functional group includes at least one of an alpha-halomethyl ether, wherein a halogen moiety may be fluoro, chloro, bromo or iodo; a beta-haloethyl ether, wherein a halogen moiety may be chloro, bromo or iodo; and a halomethylaryl, wherein a halogen moiety may be fluoro, chloro, bromo, or iodo; or carboxylic acid chloride or an activated carboxylic acid (e.g., 4-nitrophenyl ester, N-hydroxysuccinimide ester, pentafluorophenyl ester, etc.). This list of modifications is not intended to be inclusive but illustrates the scope of appropriate functional groups.

Alternatively and preferably, the chemical modification to the cyclodextrins includes two or more boronic acids or two or more aldehyde functions for specific and tight binding to carbohydrates.

According to another embodiment of the present invention, there is provided a combinatorial library of compounds containing chemically reactive groups screened for selectivity and chemical reaction with the molecular structure as a target, for creating the compound of the present invention.

According to still another embodiment of the present invention, there is provided a compound for specifically reacting at any site on a target molecule, such that these sites are not limited to the conventional active site or ligand binding site of the target.

Hereinafter, the term ‘antibody mimic’ includes but is not limited to, any synthetic substance such as an MIP or a triazine derivative, or a derivative of a natural product such as a cyclodextrin or a peptide, which has been designed or selected so as to display selective affinity for a given target structure.

### BRIEF DESCRIPTION OF THE DRAWINGS

The invention is herein described, by way of example only, with reference to the accompanying drawings, wherein:

FIG. 1 is a schematic flow chart, showing illustrative preparation of “chemically reactive” molecularly imprinted polymers (MIP-based COBALT) for the selective covalent binding of hydroxyl-containing target substances, ROH;

FIG. 2 shows a schematic depiction of the preparation of a conventional cholesterol-binding, amino-containing MIP (MS50);

FIG. 3 shows a schematic view of the two MIPs, one binding non-covalently and one binding covalently, the latter being a COBALT;

FIG. 4 shows the calibration curve for cholesterol;

FIGS. 5 and 6 show the Scatchard and binding isotherm plots respectively for the third Example;

FIG. 7 is related to the preparation of crMIP-MS71;

FIG. 8 shows the IR Spectrum of MS71 (when the maximum conversion into NCO is reached);

FIG. 9 describes the overall approach for developing MIP-based COBALTS for the binding of toxic organophosphates, in which functional monomers, A, are polymerized with a large excess of crosslinker, porogen, etc, to create an MIP, B (MIP-B), that is hydrolyzed to remove the phosphonate, leaving behind complementary cavities, containing a nucleophile, X, in MIP-C, that selectively reacts covalently with DFP to form D (MIP-D). It should be noted that the nucleophile, X, in MIP-C differs from the functional group Z in the monomer as well as Y following reaction with DFP. Inclusion of other functional monomers, such as one containing a basic group to bind the HF released, was also carried out. Note, too, that the reaction of MIP-C with DFP is initially an equilibrium binding reaction, having a given Ka, which is followed by an irreversible covalent binding reaction to give MIP-D. Also, the nature of MIP-D is such that the phosphate is readily hydrolyzed to dialkyl phosphoric acid and MIP-C, which now represents a catalytic cycle;

FIG. 10 depicts representative structures of synthesized functional monomers used for the DFP-binding MIPs that were prepared,

FIG. 11 shows the synthetic scheme used for the preparation of the 4-vinylbenzaldehyde oxime phosphate and phosphonate functional monomers 3, 9, and 10;

FIG. 12 shows a calibration curve for converting percent BChe inhibition to DPFP concentration;

FIG. 13 shows a calibration curve for DCP concentration; and

FIG. 14 illustrates the approach taken for preparing widely varying COBALTs using combinatorial libraries based on cyclodextrins having isothiocyanates for covalent reaction with the target substances, in which R1, R2=phenyl, substituted phenyl, naphthyl, substituted naphthyl, etc; there is almost no limit to the variability of the structures that can be prepared by using this scheme, by adding additional R groups, using different cyclodextrin derivatives, (eg, diamines, etc) and so forth.

### DESCRIPTION OF THE PREFERRED EMBODIMENTS

The present invention is of a variety of substances which have an affinity for a desired, preselected, target substance (a small molecule; a macromolecule such as a protein, a carbohydrate, a nucleic acid, etc.; a cell; a viral particle; etc.) and which contain chemical groups that allow these substances to form strong bonds, such as irreversible covalent bonds, with the desired target substance, which as previously described may be termed COBALTs, Covalent-Binding Antibody-Like Trapping or -Trap. The present invention includes a mechanism wherein a target species (as above) is chosen and then, by synthetic chemical procedures and modifications, novel substances (COBALTs) are obtained that exhibit selective and covalent binding to the preselected target species. The uses of the COBALTs include diagnostic, analytical, therapeutic and industrial applications.

Without wishing to be limited to a single hypothesis, the binding mechanism between the COBALT and the target substance may occur as follows. An initial non-covalent complex may be pictured as forming rapidly between the COBALT and the target; other substances, different from the target substance, may be expected to either not form such complexes or alternatively to form complexes with a much shorter lifetime (in other words, their stability will be much lower) than that of the COBALT-target substance complex. The orientation of the two components, the COBALT and the target substance, in the complex is such that a chemical reaction can rapidly ensue to produce a new substance having a strong, covalent bond (or bonds) between the two initial components. Complexes formed by the COBALT and substances other than the target substance, even if they exhibit some stability, may either not have the orientation or lifetime required for covalent bond formation or the reaction rate would be expected to be far less than that between the COBALT and the desired target substance. Use of the term complex, does not rule out the possibility of more than one complex between the COBALT and the desired target substance. Indeed, for some of the applications described herein, there may be many different complexes formed, but the overall result is that the COBALTs are expected to display a preferred affinity for the desired target substance, relative to other materials, and to preferentially react chemically with the desired target substance.

Examples of situations where the chemically reactive, COBALT, approach may have important therapeutic and other applications include increased binding of a target molecule compared to conventional, noncovalently binding agents. The irreversible chemical reaction will eventually trap all targets that initially bind, whereas with conventional, noncovalent binding, once the equilibrium constant is reached, no additional target molecules may be trapped. Illustrative potential applications include assay detection at low concentrations of the target analyte or the more effective therapeutic action of a COBALT allowing lower dosages of more effective drugs.

To illustrate one advantage of the notion of a chemically reactive binding site, where the reversible binding of a ligand is followed by a covalent bond-forming chemical reaction between the receptor and the ligand, an example may be considered with a molecularly imprinted polymer (MIP). The subject of molecularly imprinted polymers has been extensively reviewed (e.g., G. Wulff, Angew. Chem., Int. Ed. Engl. 1995, 34, 1812-1832; A. G. Mayyes and K. Mosbach, Trends Anal. Chem. 1997, 16, 321-332; E. N. Vulfson, C. Alexander, and M. J. Whitcombe Chem. Brit. 1997, 33, 23-26; K. Haupt and K. Mosbach, Trends Biotechnol. 1998, 16, 468-475; Molecular and Ionic Recognition with Imprinted Polymers, ACS Symp. Ser. 703; R. A. Bartsch and M. Maeda, Eds.; American Chemical Society, Washington, D.C., 1998) and a number of patents on this topic have been issued [e.g., U.S. Pat. No. 4,127,730 (Wulff, G., Sarhan A.); U.S. Pat. No. 5,110,833 (Mosbach. K.); U.S. Pat. No. 5,630,978 (Domb, A.,); U.S. Pat. No. 5,587,273 (Yan, M. et al.); U.S. Pat. No. 5,872,198 (Mosbach, K. et al.)]. All of these background art references are hereby incorporated by reference as if fully set forth herein.

For the purposes of the present example, assume that the MIP has been synthesized in the presence of the template molecule X; if the MIP binds a ligand X with an affinity (given as K_{D}) of 10⁻⁵, then at X concentrations much lower than 10⁻⁵ M, say, 10⁻⁷ M, essentially no detectable amounts of X binding can be measured; a sensor based on this MIP would not be expected to detect such low levels of analyte, X.

If the reversible (relatively weak) binding of X by the MIP to give a complex, MIP.X, were followed by a covalent-bond forming reaction between X and MIP, this MIP (a chemically activated MIP—a COBALT) would now be able to detect X, depending on the relative rate constants involved, because the concentration of irreversibly formed MIP-X gradually builds up to much higher values than the equilibrium concentration of complex MIP.X.

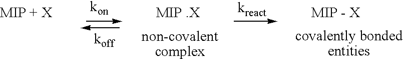

In the case of the MIPs, the system is somewhat analogous to the irreversible enzyme inhibitors and antibody affinity labeling reagents. Note that substances differing from X to an appreciable extent will tend not to react effectively within the MIP cavity because, even if they enter the MIP cavity, they will not have the orientation necessary for reaction (the stereochemical or the “spatiotemporal” [Khanjin N A, Snyder J P, Menger F M, J. Amer. Chem. Soc. 121 (50): 11831-11846 (1999)] demands are not met). The exact nature of the chemically reactive group on the “antibody mimics”, the chemically reactive “receptor”, or COBALT, whether based upon MIPs, peptides, triazines, cyclodextrins, etc., will depend on the requirements and nature of the substance to be bound. The chemical reactions presented are illustrative; other reactions are obvious to those skilled in the art and may be selected from, but are not limited to, the abundant literature on other reactions where covalent bonds are formed. Various illustrative examples of the kinds of chemical reactions that may be carried out can be found in the literature on: affinity labeling, bioconjugate chemistry, and enzyme-active site and receptor-binding site labelling reactions.

The term “enhanced apparent affinity constant” is used herein to describe the ability of the COBALT to bind more of a target substance than a conventional, non-covalently binding substance. Since affinity or binding constants are restricted to equilibrium systems, and since the use of irreversible inhibitors technically does not allow equilibrium to be reached, this definition is a generalization from a classical equilibrium system. However, the definition does permit a quantitative estimate of the improved sequestering of a given target substance to be made. Thus, if a conventional MIP, after 24 hours, or 48 hr, is determined to have bound a given fraction of a target substance, the equilibrium binding constant can be determined for this equilibrium system. If a chemically reactive MIP, or a COBALT, is determined to have bound an increased fraction of a target substance after a similar period of time has elapsed, this increased fraction may be termed “enhanced apparent affinity”, even though the COBALT is a dynamic system and the “enhanced apparent affinity constant” may vary with time.

The COBALTs comprise many different classes of laboratory-synthesized substances and also include chemically modified monoclonal antibodies, as well. In the latter case, an antibody is elicited to an appropriate hapten and then the antibody is chemically “activated” in order to convert it to a COBALT, e.g., amine to isothiocyanate; tyrosine to an o-quinone; thiol to chloromethylthioether; etc. The antibody-based COBALT will now complex with the desired target substance and subsequently bind covalently and irreversibly.

The many substances that are suitable for producing COBALTs include, but are not restricted to: (i) molecularly imprinted polymers (MIPs), which have been prepared in one of the conventional manners reported in the literature, and then chemically converted to an “activated MIP”, i.e., a COBALT, which specifically binds to and then reacts, forming a covalent bond with the target substance; (ii) peptides, e.g., cyclic peptides, which have been modified to contain a reactive functional group, such as an isothiocyanate group, so that the peptide will not only bind to a specific target substance but covalently react with that substance; (iii) peptide-derivatives of “platform” molecules, such as cyclodextrins, where the peptide or cyclodextrin has been chemically activated to specifically bind to, and covalently react with, a desired target substance; (iv) non-peptide substances, such as triazine derivatives, which, again, have affinity for and chemically react with a given target substance; (v) non-peptide-derivatives, such as triazine derivatives, of molecules, such as cyclodextrins, where either the triazine derivative or the cyclodextrin has been chemically activated (or where both have been activated) to specifically bind to and covalently react with, a desired target substance. The target substances are not restricted to any class of material and include but are not limited to, small molecules such as steroids, sugars, lipids; macromolecules such as proteins, carbohydrates and nucleic acids; cell surface substances and receptors; and other molecules of biomedical interest. The invention particularly relates to the use of COBALTs as drugs and for detection as well as separation applications.

Production of the COBALT may include, but is not limited to, the following three approaches, which are given only as non-limiting illustrative examples:

i. Creating a compound having selectivity for the target substance (e.g., an MIP) and then ‘activating’ the compound by introducing a chemically reactive functional group at an appropriate, specific locus on the compound in order for covalent bond reaction to take place when the target molecule is in contact with the compound. Alternatively, if the target substance has a reactive functional group, e.g., an ester, epoxide, disulfide, etc. or fluorophosphates or fluorophosphonate, as in the case of chemical warfare agents, the MIP or other COBALT can optionally be prepared such that an appropriate functional group is present to react with the target.

ii. Chemically modifying a substance, known to be selective for the target material, so that it reacts covalently with the target; this substance may also react at a number of different sites on the target substance.

iii. Creating a combinatorial library of related or different COBALTs, designed for both selectivity and chemical reactivity; these are then screened for optimum performance with the target substance.

In addition, in a manner that is reminiscent of the evolution of improved binding in antibodies produced by the immune system, the COBALTs may be further “evolved”, i.e., chemically changed and further selected in order to obtain improved binding-plus-reacting-substances for the given, desired target material.

In the previous description, the COBALT has been described as having one reactive group for covalent bond formation with the target substance, but, as illustrated below, two or more activated functional groups may be present on each COBALT.

A large variety of suitable functional groupings that can be incorporated so as to convert a substance (such as an MIP, a cyclodextrin derivative, etc.) to a COBALT. The examples given below are illustrative and not meant to limit the classes, number or examples.

| Functional group(s) on the | | Functional groups on the target |
|---|---|---|
| potential COBALT having a | | substance that enter into |
| degree of affinity for the | Functional group | covalent bond-forming reactions |
| target substance | on the COBALT | with the COBALT |
| —NH₂ | —NCO —NCS | —NH₂ —OH —SH |
| —OH | —O—CH₂—Cl | —NH₂ —OH —SH —CO₂H |
| —OH | —O—CH₂ CH₂—Cl | —NH₂ —OH —SH —CO₂H |
| methylphenyl (tolyl) | chloromethylphenyl | NH₂ —OH —SH —CO₂H |
| | | conjugated diene (Diels-Alder reaction) |
| 2, 5-diketo-N-phenyl- | 2, 5-diketo-N-phenyl- | |
| triazoline | triazoline | |
| —CO₂H | —COCl or active ester | —NH₂ —OH —SH |

Although boronic acids were used in one of the first examples of an MIP, tighter binding of carbohydrates, sugars, glycolipids, etc. may be achieved using two or more boronic acid derivatives. Similarly, acetals and ketals have been used in MIPs for binding to diols, but the use of two or more aldehydes has not been reported as for the present invention.

There are numerous examples of previously reported covalent-bond forming reactions but these examples all differ from the present invention. Chemically activated ligands have been used as enzyme inhibitors (some irreversible inhibitors form covalent bonds with the enzyme active site residues; “suicide” inhibitors; etc.), and specific labelling agents for receptors and for antibodies; the latter two are often called affinity labelling approaches.) The goals in these studies have invariably been to obtain information regarding the labelled amino acids, the binding site structure and topology, etc., but some of the irreversible enzyme inhibitors are important drugs, e.g., Orlistat.

The present invention may be differentiated from the above examples, in that, in an important and major embodiment of the invention, any site on the target substance is available for targeting by the chemically reactive enhancement is designed. This is reminiscent of the immune system's approach: antibodies elicited to, say, an enzyme, may bind at any point on the surface of the enzyme, and, when characterized, are found to be specific for a given site on the enzyme, including but not restricted to the enzyme active site. So, too, the chemically reactive, covalently binding compounds of the present invention have a priori random site-selectivity. Thus, the present invention is not dependent on the availability of a known or defined ligand binding site, or any ligand binding site at all, which is an essential requirement in all of the above approaches.

The COBALT approach enables feasible structures having chemically reactive groups, such as an isothiocyanate, to be designed or discovered, for which the desired binding plus covalent bond-forming reaction (one that may react at any site on the target substance) occurs. Although the design or discovery of such compounds according to the present invention may exploit any knowledge available regarding the target structure to improve probability of discovering effective binders, the approach of the present invention is primarily and preferably a discovery process which is dependent, as with the antibodies of the immune system, on using a large number and variety of potential binding structures.

In addition, the systems associated with the present invention (COBALTs based upon molecularly imprinted polymers (MIPs), cyclodextrins, peptides, triazines, etc.) have not been previously disclosed in the literature. There are references to peptide derivatives that form covalent bonds with enzymes but these are enzyme inhibitors of the type mentioned above. Triazine derivatives have been developed for selective binding but these have been based upon equilibrium, non-covalent binding [e.g., “Design, Synthesis and Application of a Protein A mimetic.” R-X Li, V. Dowd, D. J. Stewart, S. J. Burton and C. R. Lowe (1998) Nature Biotechnologyl 16,190-195].

### EXAMPLE 1

### Cholesterol-binding MIP and Cholesterol-binding MIP-based Cobalt Bile Acid-binding and Bile Acid-binding MIP-based Cobalts

This Example relates to the design and implementation of COBALTs for binding cholesterol and bile acids.

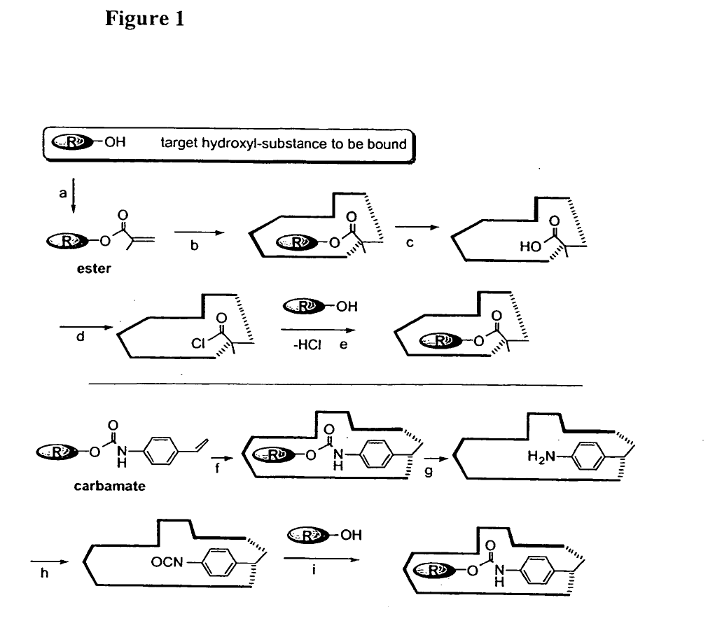

Cholesterol, whose efficient, specific elimination from the body represents an important therapeutic advance in atherosclerosis and deoxycholic acid (DCA), a toxic bile acid produced by bacteria present in intestinal flora, represent important applications. FIG. 1 shows a schematic outline of the approach for the creation of specific COBALTs.

As shown in FIG. 1, the hydroxyl-containing cholesterol and DCA targets are represented by R—OH. An ester R—O—CO—R′ or carbamate R—O—C(═O)—NH—R′ derivative of the target alcohol ROH is prepared where the R′ group has a vinyl, polymerizable function such as styrene, methacrylyl, etc. The ester or carbamate is then polymerized with a large excess of crosslinker in the conventional fashion [G. Wulff and A. Sarhan Angew. Chem. Int. Ed. Engl. 11, 341-(1972); G. Wulff. Angew. Chem. Int. Ed. Engl. 34, 1812-1832 (1995); G. Wulff, et al. Angew. Chem. Int. Ed. Eng. 36, 1962-1964 (1997)] to give a highly crosslinked polymer containing many “buried” copies of the R—O—C(═O)— function. The polymer is then treated chemically (MeOH/KOH/H₂O solution, for example) to hydrolyze essentially all of the ester or carbamate bonds, releasing the R—OH imprint molecule and creating selective cavities containing a carboxylate (for the ester derivative) or an amino group (for the carbamate derivative). Typically, approximately 80-95% of the “template” groups R—OH are thus removed.

More specifically, as shown, the top portion of FIG. 1 utilizes polymerization of an ester derivative of ROH to give, after hydrolytic removal of the print or template molecule ROH, carboxylic acid-containing cavities complementary to ROH which are activated to acid chlorides in order to form the covalent product of step e. The bottom portion exemplifies polymerization of a carbamate derivative of ROH to afford, following removal of ROH, complementary amine-containing cavities, which are activated to isocyanate (or isothiocyanate, not illustrated here) to form the covalent product, step i. All of the steps are routinely used in MIP technology: step a, synthesis of the methacylic ester of ROH (synthesis of the carbamate from 4-vinylbenzeneisocyanate is not illustrated); steps b and f, the ester or carbamate is polymerized with a large excess of crosslinker and a porogen (pore-forming solvent) using an initiater such as AIBN and heat (or irradiation); steps c and g, the solid polymer is ground, sieved and treated with reagents to hydrolzye all ester and carbamate bonds and remove ROH; step d, the acid chloride MIP-based COBALT may be prepared by treating with SOCl₂ or COCl₂; step h, the isocyanate (or isothiocyanate) MIP-based COBALT can be prepared by reaction with phosgene, COCl₂. (or thiophosgene, CSCl₂).

The resulting MIPs may be used directly as conventional binding agents or they may be converted into COBALTs by chemical modification, or “activation”, using specific chemical reactions. The carboxylate polymers (derived from the ester derivative) are chemically converted to acid chloride, Cl—C(═O)-polymer, while the amino polymers (derived from the carbamate derivative) are converted to isocyanate, O═C═N-polymer, or isothiocyanate, S═C═N-polymer. Since the different reactive functional groups will have varying reactivities and orientations and distances from the target molecules, a family of many MIP-based COBALTs may be prepared and tested to find the materials having optimal performance.

Experiments carried out on the resulting MIP-based COBALTs show that the apparent equilibrium binding constants of R—OH to the COBALTs are much higher than with the parent MIPs; this difference is especially pronounced as the concentration of R—OH is decreased and incubation time is increased.

When the two MIPs, the covalently bound (COBALTs) and non-covalently bound are treated under conditions which dissociate R—OH from the latter, the former remain bound to R—OH.

### EXAMPLE 2

### Steroid MIPs

The COBALT approach is illustrated using chemically reactive molecular imprint polymers (MIPs). Cholesteryl 4-vinylphenyl carbamate was used as a template monomer; cholesteryl methacrylate was an added functional monomer to create hydrophobic binding and recognition. Cholesterol was cleaved from the polymer (MS40, MS41) hydrolytically with the concomitant loss of CO₂, resulting in the formation of a conventional MIP (MS50) having a non-covalent (or non-reactive) recognition site, bearing an aminophenyl group, capable of interacting with cholesterol through hydrogen bonding. By chemical modification of the polymer with phosgene and thiophosgene, respectively, the amino group was transformed into reactive isocyanate (MS71) and isothiocyanate (MS80) groups, respectively, both of which bound the cholesterol in a covalent fashion.

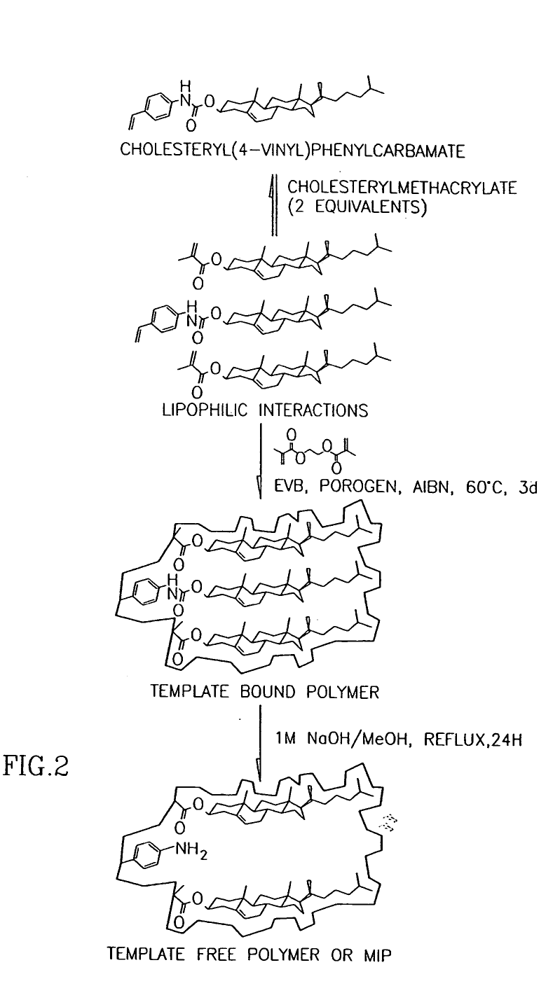

FIG. 2 shows a schematic depiction of the preparation of a conventional cholesterol-binding, amino-containing MIP (MS50). In separate experiments it was shown that a polymer of cholesteryl methacrylate is completely resistant towards hydrolysis (no release of cholesterol under more drastic hydrolysis conditions than used to obtain MS50); cholesteryl methacrylate was therefore used as a functional monomer to obtain a lipophilic recognition cavity.

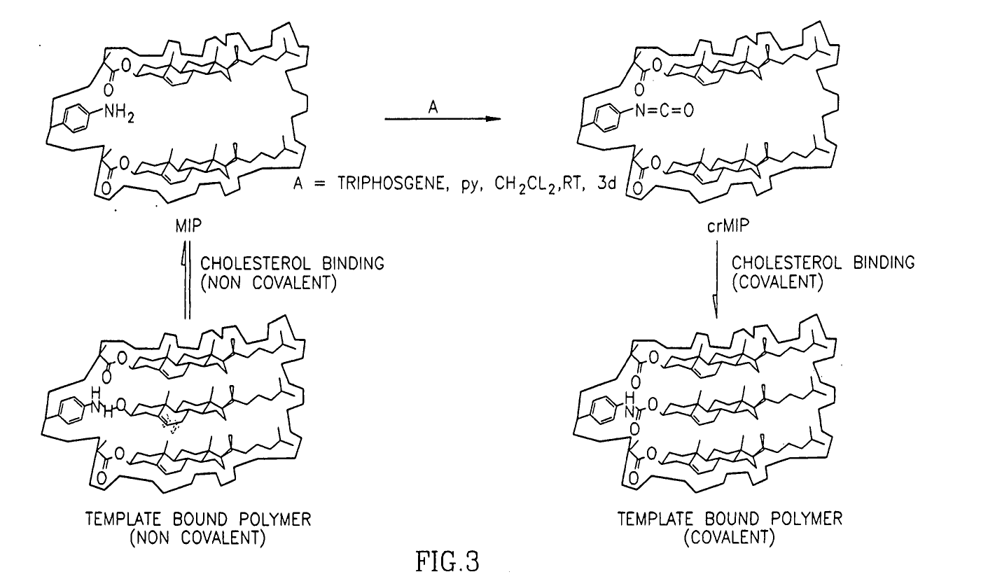

FIG. 3 shows a schematic view of the two MIPs, one binding non-covalently and one binding covalently, the latter being a COBALT. The isocyanate MIP (MS71) is illustrated; the analogous reaction using thiophosgene afforded the isothiocyanate MIP (MS80).

Cholesterol Methacrylate

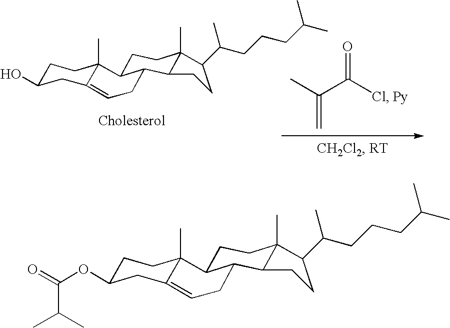

To a mixture of cholesterol (1.0 g, 2.60 mmol) and pyridine (0.25 ml, 3.12 mmol) in dichloromethane (10 ml) containing ca. 0.01 g hydroquinone, was added methacryloyl chloride (0.3 ml, 3.12 mmol) drop wise at 0° C. The mixture was stirred at RT for 4 h transferred to a separatory funnel, washed with saturated aq. sodium bicarbonate solution (2×10 ml) followed by brine (2×10 ml). The organic layer was dried over anhyd. sodium sulphate, and the solvent evaporated in vacuo. The residue (1.15 g, 97%) was recrystallized from hot ethylacetate using ca. 0.05 g of hydroquinone, which afforded long needles of cholesteryl methacrylate (1) (1.0 g, 84%), m.p. 98-100° C.

¹H NMR (300 MHz, CDCl₃): δ (delta) 0.63 (s, 3H, 18-CH₃), 0.83 (d, 6H, 27-CH₃, J=7.7 Hz), 0.86 (d, 3H, 21-CH₃, J=6.9 Hz), 1.02 (s, 3H, 19-CH₃), 0.88-2.05(br, steroid nucleus), 1.89 (s, 3H, allylic CH₃), 2.38 (d, 2H, J=8.5 Hz, C7-H₂) 4.66 (m, 1H, 3α-H), 5.40 (m, 1H, C6-H), 5.48 (s, 1H, ═CH anti to allylic CH₃), 6.30 (s, 1H, ═CH syn to allylic CH₃).

4-Vinylbenzazide

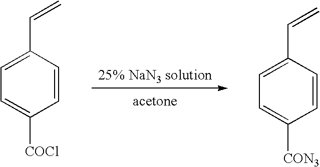

Freshly distilled 4-vinylbenzoylchloride (1.2 g, 7.2 mmol) was dissolved in dry acetone (25 ml) containing 0.1 g of hydroquinone and cooled to 0° C. A cold solution of sodium azide (4.68 g, 72 mmol) in water (20 ml) was slowly added to the acid chloride solution. The reaction mixture was stirred at RT for 2 h, extracted with dichloromethane (3×20 ml) and the solvent evaporated. The crude product was recrystallized with ethyl acetate by adding a pinch of hydroquinone (yield, 1.07 g, 86%), m.p. 167-169° C.

¹H NMR (300 MHz, CDCl₃): δ (delta) 5.40 (d, 1H, J=11.7 Hz, vinyl C2-H, cis), 5.84 (d, 1H, J=19.5 Hz, vinyl C2-H, trans), 6.67 (dd, 1H, J_{1 and 2}=19.5 and 11.7 Hz, vinyl C1-H), 7.44 (d, 2H, J=9.7 Hz, aromatic, ortho to vinyl group), 8.00 (d, 2H, J=9.7 Hz, aromatic, ortho to acylazide group).

Cholesteryl(4-vinyl)phenylcarbamate

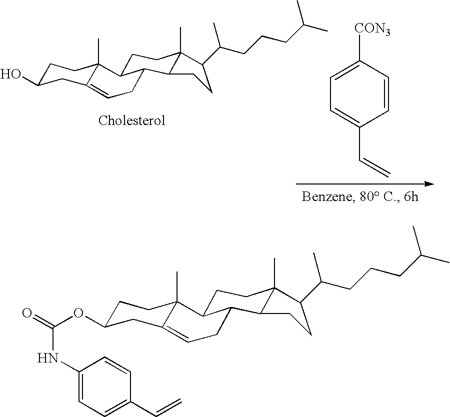

Solid 4-vinylbenzoylazide (0.495 g, 2.86 mmol) was added in five small portions with a spatula to a clear solution of cholesterol (1.0 g, 2.60 mmol) in 25 ml of benzene (dried overnight over CaCl₂) containing ca. 0.01 g of hydroquinone, preheated in an oil bath to 80° C. Slow evolution of N₂ gas was observed which subsided after 6 h. The clear reaction mixture was cooled to RT (a solid started precipitating out at this stage) and the solvent was evaporated (rotary evaporator). The crude solid was dissolved in 50 ml of dichloromethane, washed with 5% aq. sodium bicarbonate solution (3×50 ml). The solvent was evaporated and the crude was recrystallized from hot ethyl acetate using ca. 0.01 g of hydroquinone (yield, 1.33 g, 92%).m.p. 178-180° C.

¹H NMR (300 MHz, CDCl₃): [symbol] delta)0.63 (s, 3H, 18-CH₃), 0.83 (d, 6H, 27-CH₃, J=7.8 Hz), 0.86 (d, 3H, 21-CH₃, J=7.4 Hz), 1.02 (s, 3H, 19-CH₃), 0.80-2.04 (br, stroid nucleus), 2.40 (m, 2H, C7-H₂) 4.61 (m, 1H, 3α-H), 5.18 (d, 1H, J=11.7 Hz, vinyl C2-H, cis), 5.40 (m, 1H, C6-H), 5.64 (d, 1H, J=19.5 Hz, vinyl C2-H, trans), 6.58 (s, 1H, NH), 6.63 (dd, 1H, J_{1 and 2}=19.5 and 11.7 Hz, vinyl C1-H), 7.38 (s, 4H, aromatic).

Illustrative Procedure for Bulk Polymerization:

A clear solution containing cholesteryl (4-vinyl)phenyl carbamate (0.1 g) ethyleneglycol dimethacrylate (EGDM) (0.736 g), cholesteryl methacrylate (0.171 g), EVB (0.132 g) (the mole ratio of these components is 1:20:2:4), porogen (EtOH/acetonitrile, 1:1 v/v, 3 ml), and initiator, AIBN, 0.012 g, was transferred to a thick-walled glass polymerization tube. The tube was subjected to three freeze-evacuate-thaw cycles followed by flame sealing. The sealed tube was kept in an oven at 60° C. for 3 days. Following polymerization, the neck of the tube was broken and the polymer monolith was broken into small pieces using a spatula and the broken pieces ground in a mortar. The polymer was Sohxlet-extracted using 60 ml MeOH and dried in vacuo at 70° C. and then weighed. The polymer (MS41, 1.0 g) was suspended in 1M sodium hydroxide in methanol (50 ml) and heated to reflux for 24 h. periods. The cooled suspension was neutralized with 1N hydrochloric acid, filtered on a sintered glass funnel and washed with water until the washings were neutral, followed by several methanol washings. The mixture of the hydrolysate and all washings were extracted with dichloromethane (3×50 ml), dried over anhyd. sodium sulphate and evaporated in a pre-weighed flask. The purity of the resulting cholesterol was confirmed by TLC and its mass was used to calculate the degree of hydrolysis. The cholesterol removed constituted 97% of the calculated amount due to hydrolysis of the carbamate.

Control polymer was prepared in a similar way but the carbamate template was excluded from the polymerization mixture.

### EXAMPLE 3

### Tests for Cholesterol Sequestrants of Example 1

Analysis of Cholesterol

Analysis of cholesterol was performed based on its color reaction with a modified and stable Liebermann-Buchard reagent developed by Huang and co-workers.

Cholesterol (FW: 386.67)

Preparation of Liebermann-Buchard reagent: Glacial acetic acid (90 ml) was slowly added to well stirred acetic anhydride (180 ml).

Concentrated sulfuric acid (30 ml) was added to the above solution carefully at RT, which causes a slight increase in the solution temperature, followed by 6 g of anhydrous sodium sulfate. The solution was stirred vigorously for 5 min. and the clear solution was transferred to a bottle and sealed with parafilm. The bottle was stored in a refrigerator at <4° C. According to Sommers and co-workers the reagent is stable for more than a month if stored at 4° C. and they recommend not storing the reagent at RT (room temperature) longer than one day.

Preparation of stock solution of cholesterol in cyclohexane: Dissolved 0.4833 g of cholesterol in cyclohexane in a standard flask (25 ml) to obtain a 50 mM stock solution.

Standard solutions of cholesterol: From the above stock solution, 0.2, 0.4, 0.6, 0.8, 1.0, 1.2, 1.4 and 1.6 ml were withdrawn using glass syringe (1 ml) and diluted to 10 ml in standard flasks to obtain respectively solutions with concentrations of 1, 2, 3, 4, 5, 6, 7 and 8 mM. All the solutions were prepared in duplicates.

Developing cholesterol with the LB reagent: Using a glass pipette (5 ml), the LB-reagent (2.0 ml) was taken in thirteen conicle flasks (10 ml) fitted with ground glass stoppers (14′). Above standard solutions (100 μl) were added to the twelve conicle flasks containing the reagent using a micro liter syringe at RT. Solution temperature remains unchanged. Cyclohexane (200 μl) was added to the thirteenth flask containing the reagent (4 ml) which served as the reference. All the flasks were heated on a water bath at 36° C. for 15 min. After 5 min. the solutions turned to blue-green color. The flasks were removed from the water bath and allowed to cool to RT for 15 min. The OD's of the soutions were measured using UV-visible spectrophotometer at 618 nm.

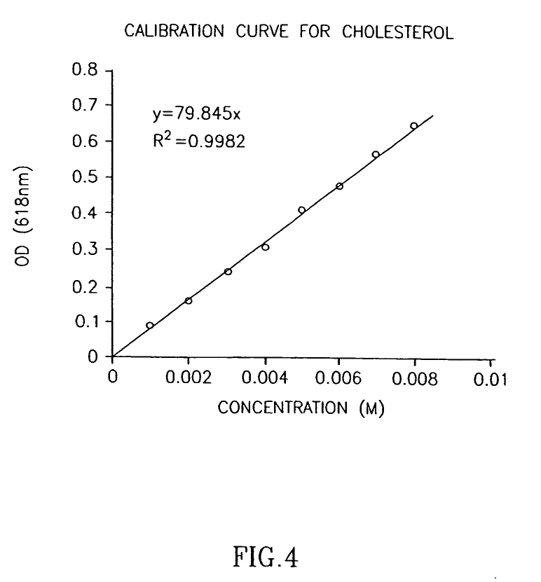

FIG. 4 shows the calibration curve for cholesterol.

Cholesterol Binding to MS50

Stock solutions of cholesterol in cyclohexane were made in 10 ml standard flasks. 1, 1.5, 2, 2.5, 3, 3.5, 4, 4.5, 5, 6, 7, 8 mM concentrations were studied in duplicates for both MS50 and its control polymer. Since only 12 conical flasks were used for the study, only three concentrations were studied at a time (six flasks each for MS50 and control polymer for three concentrations in duplicates). MS50 and control polymer were each (20 mg) weighed in 6 conical flasks fitted with ground glass joints separately. The above standard solution (2 ml) was added in the above 12 conical flask containing polymers. The flask stoppers held in place with parafilm (which also prevented introduction of moisture) and stood undisturbed overnight. After 8-10 hours of standing, samples (100 μl) were taken from the supernatant using a microlitre syringe and added directly to the LB reagent (2 ml) and proceeded with heating as mentioned in ‘Analysis of Cholesterol’. The resulting solutions were analyzed by UV-visible spectrophotometer at 618 nm. The concentrations obtained were free cholesterol concentrations which upon subtraction from initial solution concentrations gave bound cholesterol concentrations. The absorption (non-specific binding) due to control polymer is subtracted from those of MS50. Using this information, Scatchard plots were constructed. The software ‘Graphpad Prism’ was used to construct Scatchard plots.

TABLE 1
MS50 Binding Studies with Cholesterol

| | Initial | | Free | Bound |
|---|---|---|---|---|
| | Solution Conc | | Cholesterol | Cholesterol |
| Sample | (mM) | OD (618 nm) | (M) | (M) |
| MS50 | 1.0 | 0.0251 | 0.000314 | 0.000686 |
| MS50 | 1.5 | 0.0336 | 0.000421 | 0.001079 |
| MS50 | 2.0 | 0.0452 | 0.000566 | 0.001434 |
| MS50 | 2.5 | 0.0689 | 0.000863 | 0.001637 |
| MS50 | 3.0 | 0.0805 | 0.001008 | 0.001992 |
| MS50 | 3.5 | 0.0866 | 0.001085 | 0.002415 |
| MS50 | 4.0 | 0.1235 | 0.001547 | 0.002453 |
| MS50 | 4.5 | 0.1545 | 0.001935 | 0.002565 |
| MS50 | 5.0 | 0.1809 | 0.002266 | 0.002734 |
| MS50 | 6.0 | 0.2262 | 0.002833 | 0.003167 |
| MS50 | 7.0 | 0.3001 | 0.003759 | 0.003241 |
| MS50 | 8.0 | 0.3668 | 0.004594 | 0.003406 |
| MS50 | 1.0 | 0.0256 | 0.000321 | 0.000679 |
| MS50 | 1.5 | 0.0343 | 0.00043 | 0.00107 |
| MS50 | 2.0 | 0.0457 | 0.000572 | 0.001428 |
| MS50 | 2.5 | 0.0693 | 0.000868 | 0.001632 |
| MS50 | 3.0 | 0.0811 | 0.001016 | 0.001984 |
| MS50 | 3.5 | 0.0866 | 0.001085 | 0.002415 |
| MS50 | 4.0 | 0.1231 | 0.001542 | 0.002458 |
| MS50 | 4.5 | 0.1549 | 0.00194 | 0.00256 |
| MS50 | 5.0 | 0.1802 | 0.002257 | 0.002743 |
| MS50 | 6.0 | 0.2268 | 0.002841 | 0.003159 |
| MS50 | 7.0 | 0.3001 | 0.003759 | 0.003241 |
| MS50 | 8.0 | 0.3666 | 0.004591 | 0.003409 |

TABLE 2
Control Polymer Binding Studies with Cholesterol

| | Initial | | Free | Bound |
|---|---|---|---|---|
| | Solution Conc | | Cholesterol | Cholesterol |
| Sample | (mM) | OD (618 nm) | (M) | (M) |
| Control | 1.0 | 0.0713 | 0.000893 | 0.000107 |
| Control | 1.5 | 0.1008 | 0.001262 | 0.0002376 |
| Control | 2.0 | 0.1273 | 0.001594 | 0.0004057 |
| Control | 2.5 | 0.1624 | 0.002034 | 0.0004661 |
| Control | 3.0 | 0.1929 | 0.002416 | 0.0005841 |
| Control | 3.5 | 0.2223 | 0.002784 | 0.0007159 |
| Control | 4.0 | 0.2565 | 0.003212 | 0.0007875 |
| Control | 4.5 | 0.2879 | 0.003606 | 0.0008943 |
| Control | 5.0 | 0.322 | 0.004033 | 0.0009672 |
| Control | 6.0 | 0.3831 | 0.004798 | 0.001202 |
| Control | 7.0 | 0.4612 | 0.005776 | 0.0012238 |
| Control | 8.0 | 0.5293 | 0.006629 | 0.0013709 |
| Control | 1.0 | 0.0732 | 0.000917 | 0.0000832 |
| Control | 1.5 | 0.1038 | 0.0013 | 0.0002 |
| Control | 2.0 | 0.1244 | 0.001558 | 0.000442 |
| Control | 2.5 | 0.1721 | 0.002155 | 0.0003446 |
| Control | 3.0 | 0.1937 | 0.002426 | 0.000574 |
| Control | 3.5 | 0.2206 | 0.002763 | 0.0007371 |
| Control | 4.0 | 0.2505 | 0.003137 | 0.0008627 |
| Control | 4.5 | 0.2957 | 0.003703 | 0.0007966 |
| Control | 5.0 | 0.3196 | 0.004003 | 0.0009972 |
| Control | 6.0 | 0.3873 | 0.004851 | 0.0011494 |
| Control | 7.0 | 0.4599 | 0.00576 | 0.0012401 |
| Control | 8.0 | 0.5309 | 0.006649 | 0.0013509 |

TABLE 3
Correction of Non-specific Binding

| | | Control | | |
|---|---|---|---|---|
| | | Polymer | Net Cholesterol | |
| | | bound | bound by MS50 | Net Cholesterol |
| Initial | | Cholesterol | (MS50 | bound by |
| Solution | MS50 bound | Conc (M) | bound-Control | MS50/Free |
| Conc | Cholesterol | (non-specific | Polymer bound) | Cholesterol |
| (mM) | Conc (M) | binding) | Conc (M) | (Bound/Free) |
| 1.0 | 0.000686 | 0.000107 | 0.000579 | 1.840701 |
| 1.5 | 0.001079 | 0.0002376 | 0.000841 | 1.99894 |
| 2.0 | 0.001434 | 0.0004057 | 0.001028 | 1.815773 |
| 2.5 | 0.001637 | 0.0004661 | 0.001171 | 1.356992 |
| 3.0 | 0.001992 | 0.0005841 | 0.001408 | 1.396342 |
| 3.5 | 0.002415 | 0.0007159 | 0.001699 | 1.566842 |
| 4.0 | 0.002453 | 0.0007875 | 0.001665 | 1.076617 |
| 4.5 | 0.002565 | 0.0008943 | 0.001671 | 0.863412 |
| 5.0 | 0.002734 | 0.0009672 | 0.001767 | 0.780071 |
| 6.0 | 0.003167 | 0.001202 | 0.001965 | 0.693618 |
| 7.0 | 0.003241 | 0.0012238 | 0.002017 | 0.53677 |
| 8.0 | 0.003406 | 0.0013709 | 0.002035 | 0.443 |
| 1.0 | 0.000679 | 0.0000832 | 0.000596 | 1.859449 |
| 1.5 | 0.00107 | 0.0002 | 0.00087 | 2.026195 |
| 2.0 | 0.001428 | 0.000442 | 0.000986 | 1.722068 |
| 2.5 | 0.001632 | 0.0003446 | 0.001287 | 1.483375 |
| 3.0 | 0.001984 | 0.000574 | 0.00141 | 1.388458 |
| 3.5 | 0.002415 | 0.0007371 | 0.001678 | 1.547387 |
| 4.0 | 0.002458 | 0.0008627 | 0.001596 | 1.034912 |
| 4.5 | 0.00256 | 0.0007966 | 0.001763 | 0.90896 |
| 5.0 | 0.002743 | 0.0009972 | 0.001746 | 0.773605 |
| 6.0 | 0.003159 | 0.0011494 | 0.00201 | 0.707655 |
| 7.0 | 0.003241 | 0.0012401 | 0.002001 | 0.532487 |
| 8.0 | 0.003409 | 0.0013509 | 0.002058 | 0.448165 |

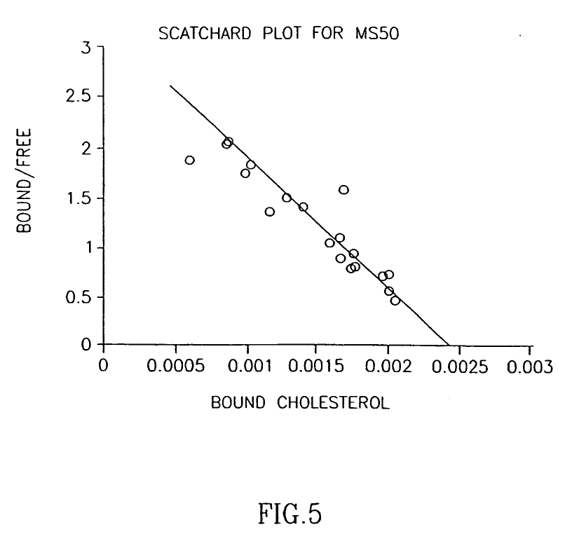

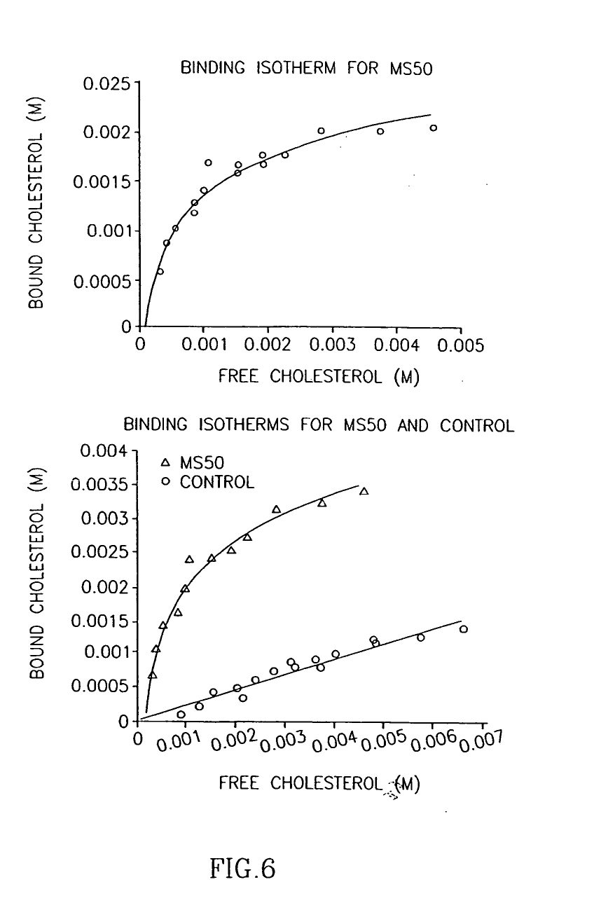

FIGS. 5 and 6 show the Scatchard and binding isotherm plots respectively for this Example.

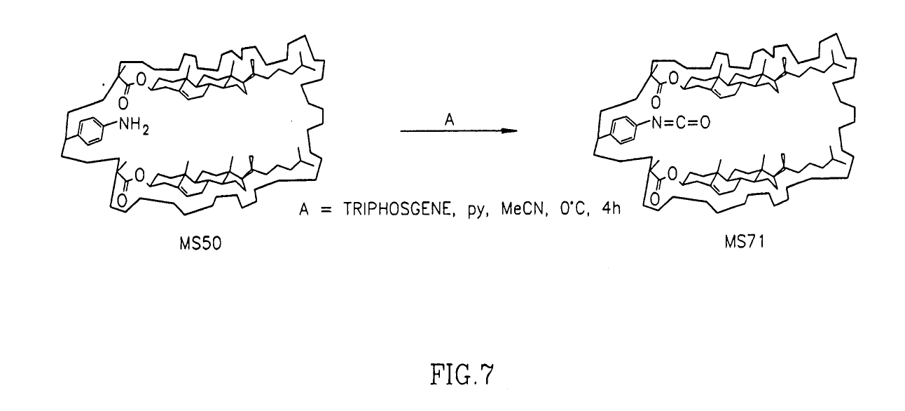

FIG. 7 is related to the preparation of crMIP-MS71.

To a suspension of MS50 (0.5 g˜0.1 mmol of NH₂ groups) in dry MeCN (50 ml) was added 4.947 g of triphosgene (16.66 mmol) at once. The resulting mixture was stirred for 30 min. and cooled to 0° C. using ice-salt mixture. Pyridine (4 ml, 50 mmol) was added to the suspension slowly using a syringe, over a period of one hour. The contents were stirred at this temperature for 4 h. the reaction was monitored by IR spectroscopy following the developing NCO stretching at ν=2265 cm⁻¹ (nujol). The suspension was filtered and the polymer was washed with dry acetonitrile (2×10 ml) and dried in the oven at 80° C. for 30 min. The resulting polymer MS71 was further dried under vacuum for 1 h and stored in a desiccator. During filtration and drying processes, the NCO peak % T at 2265 cm-1 reduced to 50% compared to that measured from the reaction mixture. Moreover, upon standing in the atmosphere for overnight, the NCO peak disappeared completely.

Binding studies of COBALT MS71 compared to conventional MIP MS50

| | Initial | | | | |
|---|---|---|---|---|---|
| Polymer | Conc | OD | Free | Bound | Bound |
| (20 mg) | (M) | (618 nm) | (M) | (M) | (mg) |
| MS71 | 0.005 | 0.1099 | 0.001376 | 0.003624 | 2.8 |
| MS71 | 0.005 | 0.1245 | 0.001559 | 0.003441 | 2.66 |
| MS50 | 0.005 | 0.187 | 0.002342 | 0.002658 | 2.05 |
| MS50 | 0.005 | 0.1779 | 0.002228 | 0.002772 | 2.14 |

In these duplicate experiments it is seen that the COBALT gave more than a 30% increase in the amount of cholesterol bound under these conditions. When the two polymers were refluxed with methanol (discarded) and then hydrolyzed and the hydrolyzate tested for cholesterol, MS50 afforded no detectable cholesterol, while MS71 afforded nearly the amount taken up in the binding experiment, in accord with the formation of a stable, covalent bond in the latter, a COBALT.

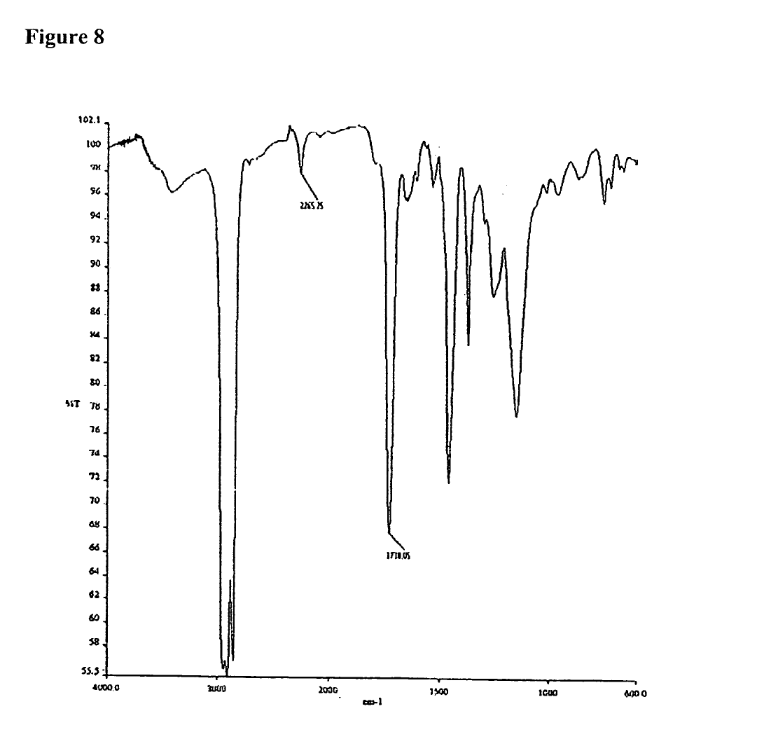

FIG. 8 shows the IR Spectrum of MS71 (when the maximum conversion into NCO is reached).

Polymer MS50 was converted in the same way to the isolhiocyanate MIP, MS80. This polymer was found to be far more stable, as expected, than the isocyanate polymer MS71. On standing for 48 hr open to the atmosphere, the decrease of the characteristic NCS peak in the infrared, at 2087 cm⁻¹, indicated that only about 50% had reacted. When treated with cholesterol as in the case of MS71, it was possible to show significant binding at concentrations where the ‘parent’ MIP was binding only very small amounts of cholesterol. At an initial cholesterol concentration of 0.005 M, MS80 (20 mg polymer samples were used in all experiments in duplicate) bound 39% more cholesterol than did MS50; at an initial cholesterol concentration of 0.003 M, MS80 bound 48% more cholesterol than did MS50; at an initial cholesterol concentration of 0.001 M, MS80 bound 76% more cholesterol than did MS50.

### EXAMPLE 4

### MIP-based Cobalts for the Binding of Toxic Organophosphates

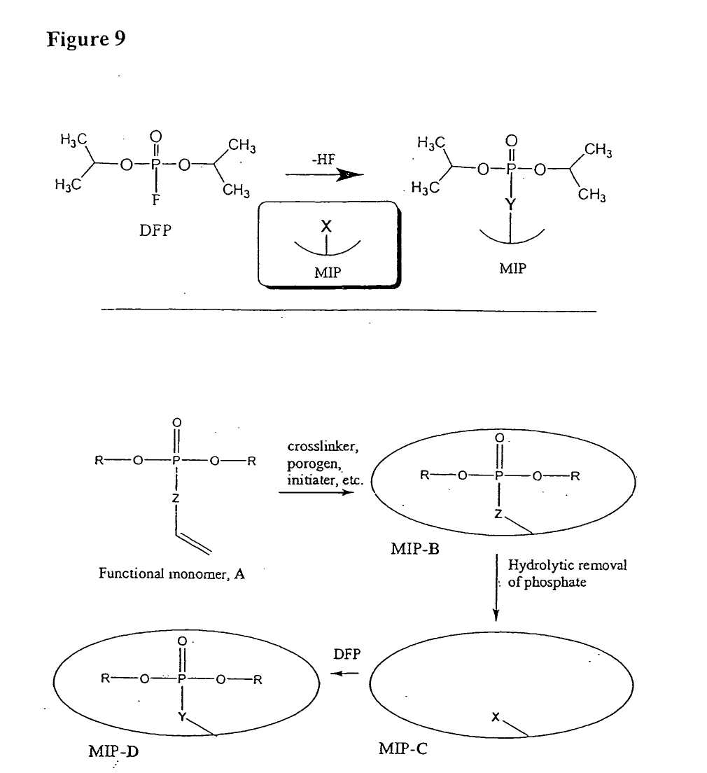

The overall approach followed is outlined in FIG. 9. DFP was chosen as a representative “OP (organophosphate) agent”, illustrating the approach that can be used for sarin, soman, etc. as well as other chemical warfare toxins. Additional “OP agents” are described below to further generalize the method. Briefly, the design was to produce MIPs having complementary cavities for DFP as well as a suitably positioned nucleophile (an active OH group that can react with DFP). The steps included: (1) the synthesis of functional monomers that contain a diisopropyl phosphate group linked to oxygen (the OH subsequently acts as the nucleophile for reaction with DFP), providing a DFP-binding cavity; (2) polymerization of the functional monomer (5-15%) with an excess (80-90%) of crosslinker and additional monomers in a solvent (porogen) that afforded pores as the macroreticular polymer formed and separated out; (3) hydrolysis of the polymer which removed diisopropyl phosphate groups, providing the selective cavity and exposing theo.oxygen nucleophile; (4) estimation of DFP binding by the MIPs. Control polymers (the unhydrolyzed polymer and/or a non-imprinted polymer prepared from the functional monomer without a diisopropyl phosphate group) were synthesized and characterized and used for comparison.

Functional Monomers.

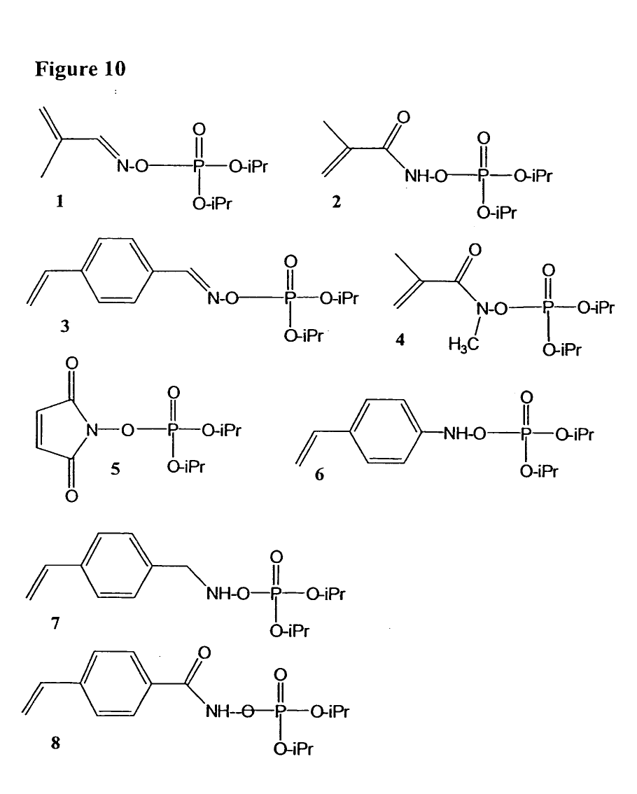

The chemistry of phosphorus compounds suggested that oxygen anucleophiles would be particularly effective in allowing hydrolysis of the phosphate groups in the activation step of the MIPs (hydrolysis of MIP-B to MIP-C; see FIG. 9) and allow good reactivity with DFP and other “OP agents” in the covalent reaction step (MIP-C to MIP-D, FIG. 9e). FIG. 10 depicts representative structures of synthesized functional monomers used for the DFP-binding MIPs that were prepared. Additional functional monomers were prepared for other fluorophosphates and fluorophosponates and representative syntheses are presented below. Each of the functional monomers was characterized (including ¹H and ³¹P NMR, MS, IR, microanalysis) and tested for thermal and hydrolytic stability for subsequent steps.

Polymerization.

Suspension polymerization (droplets of reactants dissolved in toluene suspended in water containing surface active agents were polymerized) and dispersion polymerization (also termed precipitation polymerization; solutions of reactants in a solvent—toluene/methanol—were polymerized under rapid stirring) methods were used in order to obtain relatively uniform particles and maximize the yields (conventional bulk polymerization methods, which requires subsequent grinding and sieving and results in large losses, were also used). Thermally initiated polymerization and photochemically initiated polymerization methods were also used.

Divinylbenzene/styrene (DVB/S) and ethylene glycol dimethacrylate/monomethyl acrylate (EGDMA/MMA) crosslinker/monomer mixtures werb used. Mixtures of divinylbenzene/styrene/4-vinylpyridine (DVB/S/VP) and other combinations were also used.

The organic solvent wash after polymerization was analyzed for phosphorus-containing substances (³¹P-NMR) to indicate the proportion of functional monomer (typically >80%) that had been incorporated into the polymer. The MIP polymers were also analyzed for phosphorus by the sensitive ICP method after a weighed sample for fully combusted in oxygen using a Schoeniger flask. The phosphorus content was determined by inductively coupled plasma atomic emission spectrometry (ICP-AES) at 178.200 nm.

All of the MIPs were tested for swelling in the solvent used for the binding reactions. In the divinylbenzene polymers, the size of the obtained spherical particles varied; in some suspension polymerizations ca. 50-85 [symbol] (micron) particles were obtained. In some dispersion polymerizations,1.5 to 5 [symbol] (micron) particles were obtained. Water, methanol and THF generally did not swell these polymers appreciably but toluene and 2-propanol did.

Hydrolysis of MIPs (Activation).

Polymer hydrolysis conditions were varied and optimized for MIPs. Conditions included: (1) aq. KOH/toluene/2-propanol; (2) NH₂OH—HCl/triethylamine/toluene/2-propanol; (3) NH₂OH—HCl/triethylamine/toluene/2-propano/DBU; (4) NH₂OH—HCl/40% aq. KOH/tetrabutylammonium bromide; (5) NH₂OH/toluene/2-propanol/water. Conditions (4) were generally used. In addition, extensive incubation of 3 under conditions (4) showed no chemical change to the oxime group (e.g., hydrolysis to the aldehyde).

³¹P-NMR analysis of the washes after hydrolysis of the MIP and phosphorus analysis of the polymers (ICP-AES) were used to estimate the cavity formation (activation).

OP-agent Binding to MIPs.

Assays for DFP and other OP agent binding were developed using acetylcholine esterase and butyrylcholine esterase. The calibration was carried out under conditions that allowed detection of mM equilibrium binding constants (even without any covalent binding reaction). Reactions were carried out in solution, either aqueous or organic solvent, and controls were used to assay the effects of solvent upon the enzyme.

Preparation of Functional Monomer 3:

Preparation of 4-vinylbenzaldehyde oxime: Hexamethylenetetramine (70.80 g, 50 mmol), 0.6 g hydroquinone and 4-vinylbenzylchloride (36.7 ml, 250 mmol) were mixed with stirring with glacial acetic acid (100 ml) until a solution was obtained. Water (100 ml) was added and the mixture was boiled under reflux for 2 h. Then concentrated HCl (85 ml) was added and the mixture was boiled for an additional 15 min. After cooling to room temperature the mixture was extracted with Et₂O (3×100 ml). The organic phase was washed with 10% sodium bicarbonate (3×100 ml) and with water (100 ml), dried and evaporated to yield 4-vinylbenzaldehyde (28.30 g). The latter was added to a solution of hydroxylamine (16.4 g) in 150 ml of water. The mixture was cooled to 5° C. and 10M NaOH (24 ml) was added dropwise. Stirring was continued for 48 h. Then the mixture was extracted with 2×100 ml of dichloromethane. The organic layer was washed with water, dried and evaporated to obtain the crude product (98.4% yield). The pure oxime was obtained after recrystallization from hexane and chromatography on silica gel (petroleum ether/ether eluent). ¹H NMR (CDCl₃): [symbol] delta)=5.34 (d, 1H), 5.83 (d, 1H), 6.73 (dd, 1H), 7.33-7.55 (m, 4H), 8.18 (s, 1H), 9.14 (br s, 1H).

Preparation of 3: Sodium (160 mg, 7 mmol) was added to the solution of oxime (1 g, 6.75 mmol) in dry diethyl ether (100 ml) and reaction mixture was stirred until all sodium reacted (˜6 h). A solution of diisopropylchlorophosphate (DCP) [H. McCombie, J.Chem.Soc., 1945, 380] (1.3 ml, 6.75 mmol ) in dry ether (50 ml) was added dropwise and the reaction mixture was stirred overnight at room temperature. After filtration and evaporation, the residue was chromatographed on silica gel to give monomer 3 as an oily residue. ¹H NMR (CDCl₃): [symbol] delta)=1.29 (d, 6H), 4.57 (q, 1H), 5.39 (d, 1H), 5.83 (d, 1H), 6.63 (dd, 1H), 7.44 (d, 2H), 7.55 (d,2H), 9.80 (s, 1H). ³¹P NMR (CDCl₃): [symbol] delta)=−2.39.

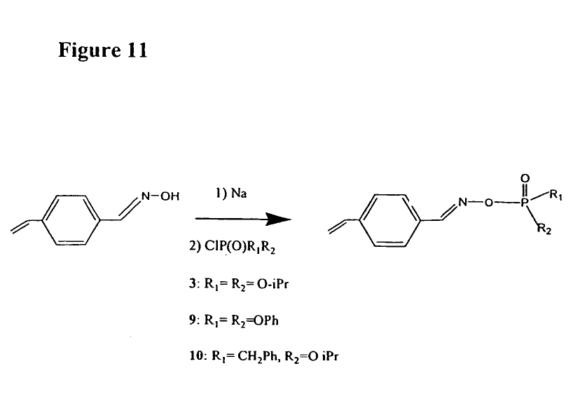

Preparation of functional monomer 9: The reaction was carried out as described for 3 using 4-vinylbenzaldehyde oxime and commercial (Aldrich) diphenylchlorophosphate; the product had m.p. 68-72° C. (see FIG. 11 for the synthetic scheme). ¹H NMR (CDCl₃): [symbol] delta)=5.37 (d, 1H), 5.84 (d, 1H), 6.72 (dd, 1H), 7.21-7.36 (m, 10H), 7.44 (d, 2H), 7.62 (d, 2H), 8.33(s, 1H). ³¹P NMR (CDCl₃): [symbol] delta)=−12.17

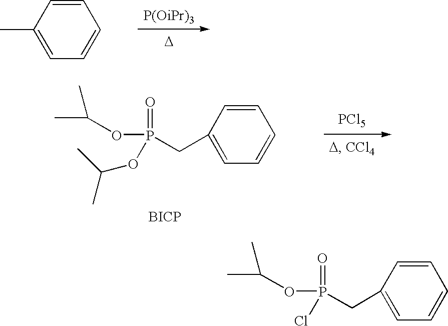

Preparation of monomer 10

The benzylisopropylchlorophosphate (BICP) was prepared by heating a mixture of triisopropylphosphite (10 ml, 60 mmol) and benzylbromide (4.76 ml, 60 mmol ) for 2 h at 90° C. The unreacted starting materials were distilled out and the residue (8.7 g) was dissolved in CCl₄ (5 ml) and heated to reflux. Phosphorus pentachloride (7.0 gr ) was then added in small portions during 1 h and the reaction mixture was refluxed for an additional 0.5 h. Distillation (0.5 mmHg) provided compound BICP (65% yield) as a colorless liquid. ¹H NMR (CDCl₃): [symbol] delta=1.25 (d, 3H), 1.31 (d, 3H), 3.44 (d, 2H), 4.83 (m, 1H), 7.28 (m, 5H). ³¹P NMR (CDCl₃): [symbol] delta)=35.68.

The synthesis of 10 was carried out as described for 3 and 9, using BICP and 4-vinylbenzaldehyde oxime. ¹H NMR (CDCl₃): [symbol] delta)=1.27 (d, 3H), 1.31 (d, 3H), 3.40 (d, 2H), 5.33 (d, 1H), 5.83 (d, 1H), 6.71 (m, 1H), 7.25 (m, 5H), 7.41 (d, 2H), 7.62 (d, 2H), 8.23 (s, 1H). ³¹P NMR (CDCl₃): [symbol] delta)=28.10.

Preparation of diphenylfluorophosphate (DPFP) and benzyl isopropylfluorophosphonate (BIFP).

Sodium fluoride (4 equivalents) was added to one equivalent of chlorodiphenylphosphate or benzylchloroisopropylphosphonate (BICP) dissolved in CCl₄ and the reaction mixture was heated to reflux for 24 h. The mixture was then filtered, an additional portion of NaF was added to the filtrate which was then heated to reflux for additional 24 h and monitored by ³¹P and by ¹⁹F NMR. The reaction mixture was filtered and evaporated. In the case of FP(O)(OiPr)CH₂Ph the product was distilled.

NMR data of FP(O)(OPh)₂: ³¹P NMR (CDCl₃): [symbol] delta)=−23.04. ¹⁹F NMR (CDCl₃): [symbol] delta)=−82.4 (d, J_{P-F}=1166 Hz)

NMR data of FP(O)(OiPr)CH₂Ph: ¹H NMR (CDCl₃): [symbol] delta)=1.81 (d, 3H), 1.52 (d, 3H), 3.21 (d, 2H), 4.78 (m, 1H), 7.28 (s, 5H). ¹³C NMR (CDCl₃): [symbol] delta)=23.63, 23.99, 31.42-33.64 (d), 73.79, 127.61, 127.66, 128.94, 129.73, 129.98. ³¹P NMR (CDCl₃): [symbol] delta)=27.14 (d). ¹⁹F NMR (CDCl₃): [symbol] delta)=−70.75 (d, J_{P-F}=1333 Hz).

Preparation of Polymer [MIP92-42] using Monomer 9
by Solution Polymerization

| | | |
|---|---|---|
| Divinylbenzene | 7.1 | g |
| Styrene | 0.7 | g |
| Toluene | 6.3 | g |
| Methanol | 23.5 | ml |
| Monomer 9 | 0.40 | g |
| Initiator 2,2′-azobisisobutyronitrile | 0.08 | g |

All ingredients were placed in a three-necked round-bottom 100 ml flask, equipped with a mechanical stirrer, condenser (CaCl₂ tube) and dropping funnel, except for the initiatordissolved in 2.7 ml methanol. The stirred solution was heated to reflux and after 0.5 hr the initiator solution was added. After an additional 6 hours reflux the reaction mixture was cooled. The colorless solid polymer beads were filtered and washed once with toluene-methanol (1:1) mixture (300 ml) and twice with acetone (300ml each). The polymer was dried under vacuum at 60° C. for 1 8 hrs. Yield=4.5 g, 55%.

Preparation of Polymer 8 MIP92-42III]

MIP92-42, 1 g, was stirred with 50 ml toluene for 0.5 hr; 5 ml of 2-propanol were then followed by 10 ml of an aqueous NaOH solution (40 g in 100 ml water). While stirring vigorously, 2.5 g of solid tetrabutylammonium bromide (same results were obtained using the chloride) were added and the stirred reaction was warmed to 70° C. for 12 hr. The cooled reaction was filtered on a sintered-glass filter (the filtrate was used to determine phosphorus content), and the polymer washed with water (50 ml), 0.1 N HCl (50 ml), 1% Na₂CO₃ solution (50 ml), six portions of 2-propanol (each 50 ml), chloroform (50 ml), three portions of toluene (50 ml), and finally dried at 60° C. for 12 hr.

Representative Binding Experiments, Using MIP 92-42III as an example.

MIP92-42III is designed to bind diphenylfluorophosphate (DPFP); the unhydrolyzed MIP 92-42 is the control polymer.

The concentrations of DPFP present during various stages of the experiment were determined by measurement of percent inhibition of butyryl choline esterase (BChe) activity. A calibration curve was constructed, using fresh solutions of DPFP in isopropyl alcohol, to convert percent BChe inhibition to DPFP concentration. BChe activity was measured in units of change in A412 per minute based on the production of thiocholine from the enzymatic hydrolysis of butyryl thiocholine, which created a yellow color in the presence of the calorimetric reagent DTNB.

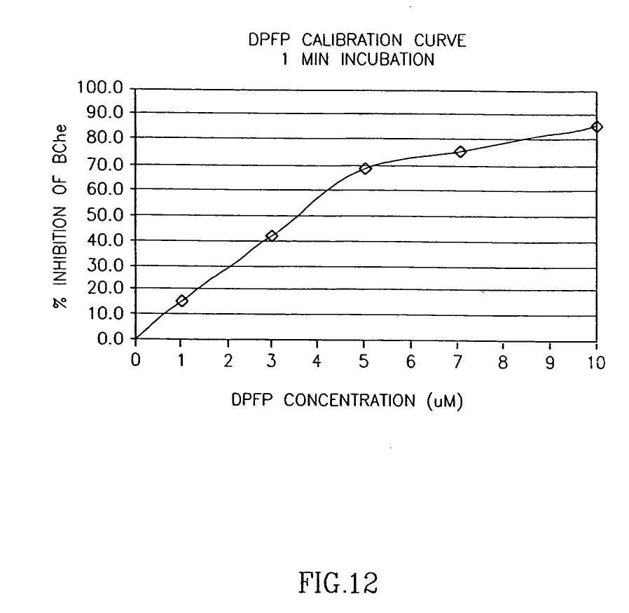

The calibration curve is shown in FIG. 12.

Samples containing 10 mg polymer, either 92-42 or 92-42III, were mixed with 1 ml isopropyl alcohol and preincubated on an oscillating shaker at room temperature for 44 hours. DPFP was then added to the samples to a final concentration of 5 μM (micromolar). The mixture of DPFP and polymer was returned to the shaker to incubate for 24 hours. The sample containing polymer 92-42 was a control for background binding that occurred in locations other than the specific binding pocket formed by the template. Additional controls containing isopropyl alcohol only (no polymer, no DPFP) and 5 μM (micromolar) DPFP in isopropyl alcohol only (no polymer) were also run. These two controls were also assayed for percent inhibition at the beginning of the incubation (time zero).

After 24 hours the samples and controls were centrifuged at 14000×g for 1 minute to sediment the polymers. The concentrations of DPFP present in the supernatants were then determined by measurement of percent inhibition of butyryl choline esterase (BChe) activity.

The percent inhibition for each sample and its equivalent DPFP concentration from the calibration curve are given in Table 1 below.

TABLE 1

| | Percent | DPFP |
|---|---|---|
| | Inhibition of | Concentration |
| Sample | BChe Activity | (μM)° |
| t = 0 hours | | |
| Control 1 isopropyl alcohol only | 0 | 0 |
| Control 2 5 μM DPFP in isopropyl | 62 | 4.5 |
| alcohol | | |
| t = 24 hours | | |
| Control 1 isopropyl alcohol only | 0 | 0 |
| Control 2 5 μM DPFP in isopropyl | 50 | 4 |
| alcohol | | |
| Control 3 5 μM DPFP + 10 mg | 57 | 4.5 |
| polymer 92-42 | | |
| Sample 5 μM DPFP + 10 mg polymer | 23 | 1.5 |
| 92-42III | | |

There was a small amount of DPFP lost due to background hydrolysis, on the order of 0.5 μM DPFP. However, the sample containing the polymer dropped significantly, to 1.5 μM, indicating that even considering a background hydrolysis of 10% of the DPFP, 60% (3 μM) of the DPFP was taken up by the polymer, clearly demonstrating the affinity of the imprinted polymer for the diphenyl compound.

Selectivity of MIP 92-42III. The above experiment was repeated using the polymers 92-42 and 92-42III with a diisopropyl phosphate cholinesterase inhibitor (diisopropyl chlorophosphate, DCP) instead of the diphenylphosphate DPFP.

Concentrations of DCP present during various stages of the experiment were determined by measurement of percent inhibition of butyryl choline esterase (BChe) activity as for DPFP above.

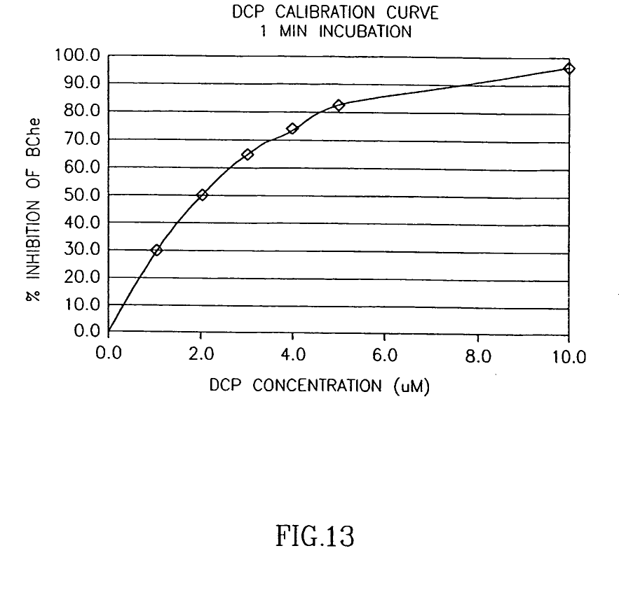

The calibration curve for DCP is shown in FIG. 13.

Samples containing 10 mg polymer, either 92-42 or 92-42III, were mixed with 1 ml isopropyl alcohol and preincubated on an oscillating shaker at room temperature for 24 hours. DCP was then added to the samples to a final concentration of 5 μM. The mixture of DCP and polymer was returned to the shaker to incubate for 24 hours. The sample containing polymer 92-42 was a control for background binding that occurred in locations other than the specific binding pocket formed by the template. Additional controls containing isopropyl alcohol only and 5 μM DCP in isopropyl alcohol only (no polymer) were run as above. In addition, controls containing 5 μM DPFP alone and DPFP with polymer 92-42III were run for comparison.

After 24 hours the samples and controls were centrifuged at 14000×g for 1 minute to sediment the polymers. The concentrations of DPFP present in the supernatants were then determined by measurement of percent inhibition of butyryl choline esterase (BChe) activity.

The percent inhibition for each sample and its equivalent DPTP concentration from the calibration curve are given in Table 2 below.

TABLE 2

| | % Inhibition | DCP |
|---|---|---|
| | of BChe | Concentration |
| Sample | Activity | (μM)° |
| t = 0 hours | | |
| Control 1 isopropyl alcohol only | 0 | 0 |
| Control 2 5 μM DPFP in isopropyl alcohol | 69 | — |
| Control 4 5 μM DCP in isopropyl alcohol | 83 | 5.0 |
| t = 24 hours | | |
| Control 1 isopropyl alcohol only | 0 | 0 |
| Control 2 5 μM DPFP in isopropyl alcohol | 52 | — |
| Control 3 5 μM DPFP + 10 mg | 6 | — |
| polymer 92-42III | | |
| Control 4 5 μM DCP in isopropyl alcohol | 72 | 4.0 |
| Control 5 5 μM DCP + 10 mg | 70 | 4.0 |
| polymer 92-42 | | |
| Sample 5 μM DCP + 10 mg | 62 | 3.0 |
| polymer 92-42III | | |

There was some DCP loss due to background hydrolysis, on the order of 1.0 μM. About an equivalent amount bound to the polymer. This amounted to 20% of the initial concentration of DCP. In comparison, almost all the DPFP (control 3) was taken up by the same amount of polymer. This lack of DCP uptake clearly demonstrates the specificity of this MIP for the diphenyl analog.

### EXAMPLE 5

### Applications of Cobalts

In addition to the previously described illustrative applications of the COBALT compounds according to the present invention, other applications are also possible. These applications may optionally include any application in which highly specific binding to a particular target molecule, followed by the formation of an irreversible covalent bond between the COBALT compound and the target molecule, is both desirable and possible. The previous description includes methods for designing and creating these COBALT compounds, which may be used according to the illustrative, exemplary applications given below.

Bile acid sequestrants. A number of polymers, such as cholestyramine, are used as bile acid sequestrants. Their action is based on the presence of strongly basic groups in the polymer (typically, ion exchange resin type of polymers) and they are used for cholesterol lowering and bile-related diseases. These materials are limited because they have limited potency and they also remove (bind) other required substances such as nutrients, drugs, etc.

Selective COBALTs according to the present invention which bind bile acids and salts do not remove needed nutrients, drugs or other substances and will be more potent. Importantly, the COBALTs can be made so that they are selective to the more hydrophobic bile acids such as deoxycholic acid. While bile acids and salts serve important functions in the body, such as promoting digestion of fat, researchers have found that the more hydrophobic (water-resistant) bile acids, such as deoxycholic acid (DCA), chenodeoxycholic acid (CDCA) and lithocholic acid (LCA) facilitate higher absorption of lipids such as cholesterol and fats into the blood stream and are toxic, causing damage to cells and promoting cancer. Current research indicates that these more hydrophobic bile acids are highly significant disease-causing agents.

The COBALTs of the present invention with their irreversible binding provide more efficient removal of bile sequestrants, with more specific binding, than compounds which are known in the art.

Environmental Detection, Removal and Protection

There is a need for detection of toxic chemicals used as weapons by the military or terrorists such as sarine or soman nerve gases. Existing biological based detectors lack stability or require special conditions for storage. This limits their application in the field. Alternatively, systems based on materials such as the cholinesterase enzymes lack selectivity to specific organo-phosphate chemical weapons (see for example USA DOD CDB02-106 Request for Proposal “Improved Field Biosensor For Organophosphates”). Similarly there is a need for compounds that remove the chemical weapons or can be used for protection e.g. a topical skin protectant (see The U.S. Army Medical Research Institute of Chemical Defense, Bioscience 2002 Medical Defense Review Conference).

MIPs have been developed to target organophosphate insecticides in water but they lack selectivity and are not usable for clean-up and protection,(see Jenkins AL et al. Analyst 126, 798-802 (2001)). The COBALTs of this invention overcome these limitations.

### EXAMPLE 6

### Cyclodextrin-based Cobalts Selected from Combinatorial Libraries

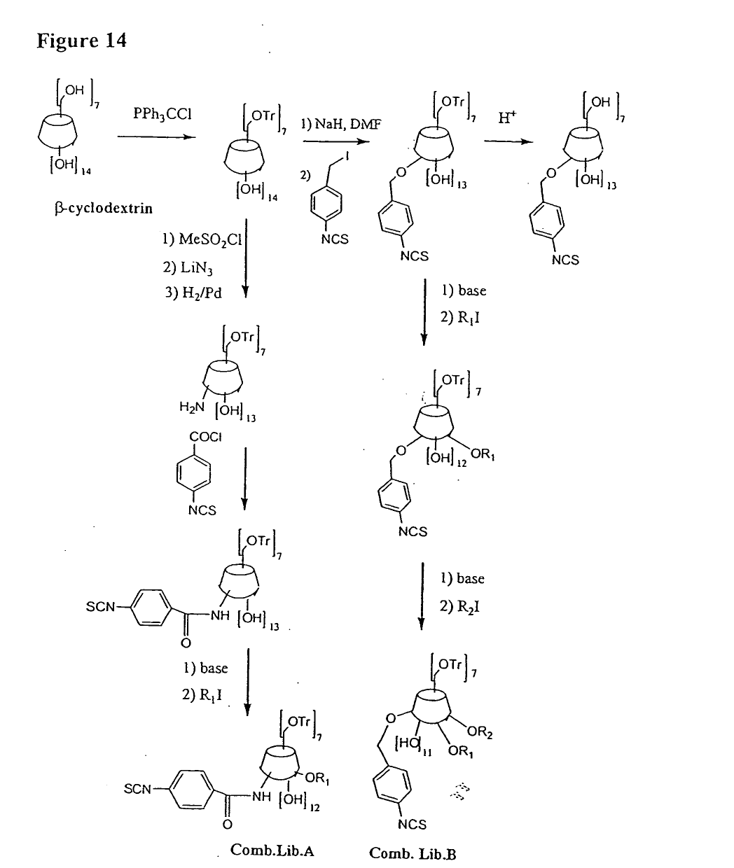

Illustrative, non-exclusive examples of approaches for obtaining COBALTs based on cyclodeextrins where the covalent bond forming group on the COBALT is an isothiocyanate are shown in FIG. 14. The beta-cyclodextrin is illustrated but alpha- or gamma-cyclodextrin based combinatorial libraries can also be used. The degree of substitution on the cyclodextrin can be varied widely; in FIG. 14 one combinatorial library (Comb.Lib.A) is illustrated with one varying substituent, R₁, while the second combinatorial library (Comb.Lib.B) is illustrated with two varying substituents, R₁ and R₂. Each could contain three, four or more substitutents, where each substituent can be a large number of different groups. In each case the final library is shown as trityl protected structures; when testing for binding the trityl groups are removed, as shown in the upper right hand comer of FIG. 14. Such COBALTs can be used for irreversible binding to small and molecules, to peptides and proteins, if these targets contain a hydroxyl or amino group.

In one example of an application of this approach, a Comb.Lib.B was prepared containing at least 1000 members. The preparation was carried out by reacting one equiv. of ten different R1I substances with tritylated-mono-4-isocyanato-benzyl-beta-cyclodextrin, in ten different tubes, each tube containing a different R1I. Each tube was further divided into ten different tubes and each reacted separately with one equiv. of R2I, again using the same set of ten different substituted benzyl iodides. After detritylation and treatrnent of each of the 1,000 tubes with one equivalent of activated fluorescent coumarincarboxylic acid, the individual substances (not totally pure materials on the basis of TLC analysis and HPLC of selected wells) were applied at different concentrations (10 microM, 1 microM, and 0.1 microM) to solutions of various proteins, including BSA, lysozyme and purified mouse IgG, and incubated at room temperature for periods of 1, 8 and 15 hrs. at various pH buffers.

There were significant differences in some of the tubes, the maximum being ca. 100- to 1000-fold differences in binding on the basis of the fluorescence. Covalent reaction, i.e., COBALT activity, was indicated by minor loss of fluorescence (<15%) after dialysis of the fluorescent protein solutions.

What is claimed is:

1. A compound for specifically binding a target molecular structure, comprising a chemically modified reactive compound that is selective for the target, having an enhanced apparent affinity constant at least double that of the chemically unmodified parent compound.

2. The compound of claim 1, wherein said compound is an antibody mimic having selective affinity for the target structure, said compound modified through chemical activation in order to react chemically with the target molecular structure.

3. The compound of claims 1 or 2, wherein said compound is a molecularly imprinted polymer (MIP), said MIP being modified through chemical activation in order to react with the target molecular structure.

4. The compound of claims 1 or 2, wherein said compound is a molecularly imprinted polymer (MIP), said MIP being modified to include a functional monomer for reacting with an activated target substance.

5. The compound of any of claims 1-4 wherein said MIP is chemically modified so as to react with the target substance to form a covalent bond, wherein said chemical modification includes at least one reactive functional group.

6. The compound of claim 5, wherein said functional group includes at least one of an isocyanate and an isothiocyanate.

7. The compound of claims 5 or 6, wherein said functional group includes at least one of an alpha-halomethyl ether, wherein a halogen moiety may be fluoro, chloro, bromo or iodo; a beta-haloethyl ether, wherein a halogen moiety may be chloro, bromo or iodo; and a halomethylaryl, wherein a halogen moiety may be fluoro, chloro, bromo, or iodo.

8. The compound of any of claims 5-7, wherein said functional group includes at least one of 2,5-diketo-N-phenyltriazoline, carboxylic acid chloride, and an activated carboxylic acid.

9. The compound of any of claims 3-8, wherein the target molecular structure is a steroid.

10. The compound of claim 9, wherein said steroid is cholesterol or a bile acid.

11. The compound of claims 1 or 2, wherein said compound is a cyclodextrin.

12. The compound of claims 1 or 2, wherein said compound is a triazine.

13. The compound of claims 1 or 2, wherein said compound is a peptide.

14. The compound of claim 13, wherein said peptide includes at least one of cyclic, linear and modified peptides and derivatives thereof.

15. The compound of any of claims 1-5, wherein the target structure is an organophosphate.

16. The compound of claim 15, wherein said compound includes at least one functional monomer for reacting with said organophosphate.

17. The compound of claims 15 or 16, wherein said compound includes a nucleophile for specifically binding to said organophosphate.

18. The compound of claim 17, wherein said nucleophile is a derivative of at least one of an oxime, a hydroxylamine, a hydrazine, a phenol and a 2-iodosobenzoic acid.

19. The compound of any of claims 1-5, wherein said compound comprises a plurality of boronic acid functions.

20. The compound of any of claims 1-5, wherein said compound comprises a plurality of aldehyde functions.

21. The compound of claims 19 or 20, wherein said functions perform specific and tight binding to carbohydrates.

22. The compound of claim 1, wherein said compound is an antibody or derivative thereof being chemically modified to react covalently with the target molecular structure.

23. The compound of any of claims 1-22, for binding to and specifically reacting with a site on the target molecular structure, apart from the active site of the target molecular structure.

24. A combinatorial library of compounds, each containing a chemically reactive group, screened for selectivity and chemical reaction with the molecular structure as a target, for creating the compound of any of claims 1-23.

25. The compound of any of claims 1-23 for use in at least one of diagnostics, combinatorial screening genomic, proteomic, and glycomic applications.

26. The compound of claim 25, wherein said combinatorial screening includes combinatorial screening for drug discovery.

27. The compound of any of claims 1-23 for use in at least one of an environmental detection, environmental removal of chemical weapons or environmental hazards and protection from chemical weapons or environmental hazards.

28. The use of compounds from any of claims 1-23 as a therapeutic compound.

29. The use of compounds of any of claims 1-23 for drugs or extracorporeal treatment.

30. A method for creating a compound for specifically binding a target molecular structure, the compound comprising a selective and chemically reactive compound with an enhanced apparent affinity constant, the method comprising:
providing a combinatorial library of compounds containing chemically reactive groups;
screening said combinatorial library for a compound having a selective chemical reaction with the target molecular structure; and
creating the compound for specifically binding the target molecular structure from at least said compound of said combinatorial library.

31. The method of claim 30, wherein said compounds in said combinatorial library include a plurality of monomers and the compound for specifically binding the target molecular structure is a polymer.

32. The method of claims 30 or 31, wherein the compound for specifically binding the target molecular structure is a MIP (molecularly imprinted polymer).

33. The method of claim 30, wherein the compound is a cyclodextrin.

34. The method of claim 30, wherein the compound is a triazine.

35. The method of claim 30, wherein the compound is a peptide.

36. The method of claim 35, wherein said peptide includes at least one of cyclic, linear and modified peptides and derivatives thereof.

37. The method of any of claims 30-36, wherein the compound features at least one functional group, said at least one functional group reacting with the target molecular structure.

38. A method for creating a compound for specifically binding a target molecular structure, the method comprising:
preparing an MIP for binding to the target molecular structure; and
converting at least a portion of said MIP to an active functional group for forming a covalent bond with the target molecular structure upon binding of said MIP to the target molecular structure, thereby forming the compound for binding the target molecular structure.

39. The method of claim 38, wherein preparing said MIP further comprises:
selecting at least one functional group of the target molecular structure;
preparing a complementary functional group for said MIP to bind to said at least one functional group; and
polymerizing a plurality of monomers containing said complementary functional group to form a polymer, wherein said polymer is said MIP.

40. The method of claim 38, wherein preparing said MIP further comprises:
selecting at least one functional group of the target molecular structure;
preparing a derivative of said target molecular structure as a functional monomer; and
polymerizing a plurality of monomers containing said functional monomer to form a polymer, wherein said polymer is said MIP.

41. The method of claim 39, wherein preparing said MIP further comprises:
hydrolyzing at least one bond of said polymer to release said target molecular structure.

42. The method of claims 40 or 41, wherein the target molecular structure is a steroid or a bile acid, and wherein said functional group of the target molecular structure is R—OH and said functional monomer includes at least one of an ester R—O—CO—R′ or carbamate R—O—C(═O)—NH—R′ derivative of said target alcohol ROH, wherein said R′ group has a vinyl, polymerizable function.

43. The method of claim 42, wherein said steroid is cholesterol.

44. The method of claim 42, wherein said bile acid includes at least one of deoxycholic acid, chenodeoxycholic acid or lithocholic acid.

45. The method of claim 42, wherein said active functional group is an acid chloride, Cl—C(═O)-polymer functional group, for reacting with a target molecular structure containing a hydroxyl group or an amino group.

46. The method of claim 42, wherein said active functional group is an isocyanate, O═C═N-polymer, or isothiocyanate, S═C═N-polymer for reacting with a target molecular structure containing a hydroxyl group or an amino group.

47. A method for creating a compound for specifically binding a chemically activated target molecular structure, the method comprising:
preparing an MIP for binding to the target molecular structure; and
converting at least a portion of said MIP to a functional group for forming a covalent bond with the target molecular structure upon binding of said MIP to the activated target molecular structure.

48. The method of claim 47, wherein preparing said MIP further comprises:
selecting at least one reactive functional group of the activated target molecular structure;
preparing a derivative of said target molecular structure which can be polymerized (a functional monomer); and
polymerizing a plurality of monomers containing said functional monomer to form a polymer, wherein said polymer is said MIP.

49. The method of claim 48, wherein preparing said MIP further comprises:
hydrolyzing at least one bond of said polymer to release said target molecular structure and leave at least one functional group in said MIP cavity for reacting with said activated target molecular structure.

50. The method of claims 48 or 49, wherein the activated target molecular structure is an organophosphate and wherein said functional monomer includes at least one of a 4-vinylbenzaldehyde oxime ester of the phosphate or phosphonate.

51. The method of claim 50, wherein preparing said MIP further comprises:
hydrolyzing at least one bond of said polymer to release said functional group of the target molecular structure.

52. The method of claims 50 or 51, wherein the target molecular structure includes at least one of cholesterol and bile acid and wherein said functional group of the target molecular structure is R—OH and said complementary functional group includes at least one of an ester R—O—CO—R′ or carbamate R—O—C(═O)—NH—R′ derivative of said target alcohol ROH, wherein said R′ group has a vinyl, polymerizable function.

53. The method of claim 52, wherein said active functional group is an acid chloride, Cl—C(═O)-polymer functional group for carbamate polymers.

54. The method of claim 52, wherein said bile acid includes at least one of deoxycholic acid, chenodeoxycholic acid or lithocholic acid.

55. The compound of claim 11, wherein said cyclodextrin is an alpha-, beta-, or gamma-cyclodextrin.

56. The compound of claim 55, wherein said cyclodextrin includes one or more amino groups for replacing one or more of the hydroxyl groups.

57. The compound of claims 55 or 56, wherein one or more hydroxyl or amino groups is linked directly to at least one of an arylcarboxylic acid group, and an arylalkylcarboxylic acid group, through an amide or ester bond.

58. The compound of any of claims 55-57, wherein one or more of the hydroxyl or amino groups is linked directly to at least one of an aryl group or arylmethyl group, wherein aryl includes at least one of phenyl and substituted phenyl, pyridyl and substituted pyridyl, naphthyl and substituted naphthyl groups.

59. The compound of claim 33, wherein said cyclodextrin is an alpha-, beta-, or gamma-cyclodextrin.

60. The compound of claim 59, wherein said cyclodextrin includes one or more amino groups for replacing one or more of the hydroxyl groups.

61. The compound of claims 59 or 60, wherein one or more hydroxyl or amino groups is linked directly to at least one of an arylcarboxylic acid group, and an arylalkylcarboxylic acid group, through an amide or ester bond.

62. The compound of any of claims 59-61, wherein one or more of the hydroxyl or amino groups is linked directly to at least one of an aryl group or arylmethyl group, wherein aryl includes at least one of phenyl and substituted phenyl, pyridyl and substituted pyridyl, naphthyl and substituted naphthyl groups.

63. The compound of claim 12 wherein said triazine is a derivatives of 2,4,6-trichloro-1,3,5-triazine (cyanuric chloride) wherein one or more of the chloro groups are replaced by an alcohol group, a phenol group or an amine group.

64. The method of claim 34 wherein said triazine includes a derivative of 2,4,6-trichloro-1,3,5-triazine (cyanuric chloride) and wherein one or more of the chloro groups are replaced by an alcohol group, a phenol group or an amine group.
